(19) United States
(12) Patent Application Publication
(10) Pub. No.: US 2024/0415840 A1
(43) Pub. Date: Dec. 19, 2024

(54) BICYCLIC AMINES AS CDK2 INHIBITORS

(71) Applicant: Incyte Corporation, Wilmington, DE (US)

(72) Inventors: Joshua Hummel, Hockessin, DE (US); Ding-Quan Qian, Newark, DE (US); Xiaozhao Wang, Moorestown, NJ (US); Ming Xiang, Wilmington, DE (US); Meizhong Xu, Hockessin, DE (US)

(21) Appl. No.: 18/736,628

(22) Filed: Jun. 7, 2024

Related U.S. Application Data
(60) Provisional application No. 63/472,153, filed on Jun. 9, 2023.

(51) Int. Cl. A61K 31/519; A61K 31/437; C07D 471/04; C07D 487/04

(52) U.S. Cl. CPC A61K 31/519; A61K 31/437; C07D 471/04; C07D 487/04

(57) ABSTRACT

The present application provides bicyclic amines of Formula (I):

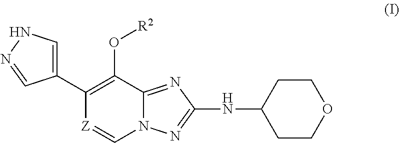

and their pharmaceutically acceptable salts thereof, that are inhibitors of cyclin-dependent kinase 2 (CDK2), as well as pharmaceutical compositions thereof, and methods of treating cancer using the same.

The present application claims the benefit of U.S. Provisional Application No. 63/472,153, filed Jun. 9, 2023, which is incorporated herein by reference in its entirety.

### SEQUENCE LISTING

This application contains a Sequence Listing that has been submitted electronically as an XML file named 20443-0822P01_SL_ST26.xml. The XML file, created on Jun. 9, 2023, is 4,859 bytes in size. The material in the XML file is hereby incorporated by reference in its entirety.

### TECHNICAL FIELD

This application is directed to bicyclic amines which inhibit cyclin-dependent kinase 2 (CDK2) and are useful for treating cancer.

### BACKGROUND

Cyclin-dependent kinases (CDKs) are a family of serine/threonine kinases. Heterodimerized with regulatory subunits known as cyclins, CDKs become fully activated and regulate key cellular processes including cell cycle progression and cell division (Morgan, D. O., Annu Rev Cell Dev Biol, 1997. 13: 261-91). Uncontrolled proliferation is a hallmark of cancer cells. The deregulation of the CDK activity is associated with abnormal regulation of cell-cycle, and is detected in virtually all forms of human cancers (Sherr, C. J., Science, 1996. 274(5293): 1672-7).

CDK2 is of particular interest because deregulation of CDK2 activity occurs frequently in a variety of human cancers. CDK2 plays a crucial role in promoting G1/S transition and S phase progression. In complex with cyclin E (CCNE), CDK2 phosphorylates retinoblastoma pocket protein family members (p107, p130, pRb), leading to de-repression of E2F transcription factors, expression of G1/S transition related genes and transition from G1 to S phase (Henley, S. A. and F. A. Dick, Cell Div, 2012, 7(1): p. 10). This in turn enables activation of CDK2/cyclin A, which phosphorylates endogenous substrates that permit DNA synthesis, replication and centrosome duplication (Ekholm, S. V and S. I. Reed, Curr Opin Cell Biol, 2000. 12(6): 676-84). It has been reported that the CDK2 pathway influences tumorigenesis mainly through amplification and/or overexpression of CCNE1 and mutations that inactivate CDK2 endogenous inhibitors (e.g., p27), respectively (Xu, X., et al., Biochemistry, 1999. 38(27): 8713-22).

CCNE1 copy-number gain and overexpression have been identified in ovarian, gastric, endometrial, breast and other cancers and been associated with poor outcomes in these tumors (Keyomarsi, K., et al., N Engl J Med, 2002. 347(20): 1566-75; Nakayama, N., et al., Cancer, 2010. 116(11): 2621-34; Au-Yeung, G., et al., Clin Cancer Res, 2017. 23(7): 1862-1874; Rosen, D. G., et al., Cancer, 2006. 106(9): 1925-32). Amplification and/or overexpression of CCNE1 also reportedly contribute to trastuzumab resistance in HER2+ breast cancer and resistance to CDK4/6 inhibitors in estrogen receptor-positive breast cancer (Scaltriti, M., et al., Proc Natl Acad Sci USA, 2011. 108(9): 3761-6; Herrera-Abreu, M. T., et al., Cancer Res, 2016. 76(8): 2301-13). Various approaches targeting CDK2 have been shown to induce cell cycle arrest and tumor growth inhibition (Chen, Y N., et al., Proc Natl Acad Sci USA, 1999. 96(8): 4325-9; Mendoza, N., et al., Cancer Res, 2003. 63(5): 1020-4). Inhibition of CDK2 also reportedly restores sensitivity to trastuzumab treatment in resistant HER2+ breast tumors in a preclinical model (Scaltriti, supra).

These data provide a rationale for considering CDK2 as a potential target for new drug development in cancer associated with deregulated CDK2 activity. In the last decade there has been increasing interest in the development of CDK selective inhibitors. Despite significant efforts, there are no approved agents targeting CDK2 to date (Cicenas, J., et al., Cancers (Basel), 2014. 6(4): p. 2224-42). Therefore it remains a need to discover CDK inhibitors having novel activity profiles, in particular those targeting CDK2. This application is directed to this need and others.

### SUMMARY

The present invention relates to, inter alia, compounds of Formula (I):

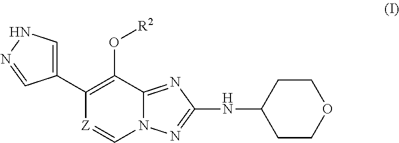

or pharmaceutically acceptable salts thereof, wherein the constituent members are defined herein.

The present invention further relates to compounds of Formula (Ia):

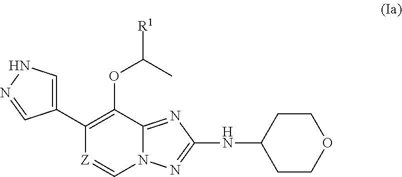

or pharmaceutically acceptable salts thereof, wherein the constituent members are defined herein.

The present invention further provides pharmaceutical compositions comprising a compound described herein, or a pharmaceutically acceptable salt thereof, and a pharmaceutically acceptable carrier.

The present invention further provides methods of inhibiting CDK2, comprising contacting the CDK2 with a compound described herein, or a pharmaceutically acceptable salt thereof.

The present invention further provides methods of inhibiting CDK2 in a patient, comprising administering to the patient a compound described herein, or a pharmaceutically acceptable salt thereof.

The present invention further provides methods of treating a disease or disorder associated with CDK2 in a patient, comprising administering to the patient a compound described herein, or a pharmaceutically acceptable salt thereof.

The present invention further provides compounds described herein, or a pharmaceutically acceptable salt thereof, for use in any of the methods described herein.

The present invention further provides uses of a compound described herein, or a pharmaceutically acceptable salt thereof, for the preparation of a medicament for use in any of the methods described herein.

### DETAILED DESCRIPTION

The present application provides, inter alia, a compound of Formula (I):

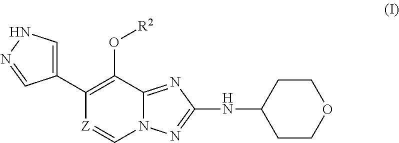

or pharmaceutically acceptable salts thereof, wherein:
Z is N or CH; and
R² is C₁₋₄ alkyl or C₁₋₄ haloalkyl.

In some embodiments, Z is N or CH; and R² is C₁₋₃ alkyl or C₁₋₃ haloalkyl.

In some embodiments, Z is N or CH; and R² is C₁₋₃ alkyl or C₁₋₃ fluoroalkyl.

In some embodiments, Z is N; and R² is C₁₋₃ alkyl or C₁₋₃ fluoroalkyl.

In some embodiments, Z is CH; and R² is C₁₋₃ alkyl or C₁₋₃ fluoroalkyl.

In some embodiments, Z is N or CH; and R² is C₁₋₃ alkyl.

In some embodiments, Z is N or CH; and R² is C₁₋₃ fluoroalkyl.

In some embodiments, Z is N; and R² is C₁₋₃ alkyl.

In some embodiments, Z is N; and R² is C₁₋₃ fluoroalkyl.

In some embodiments, Z is CH; and R² is C₁₋₃ alkyl.

In some embodiments, Z is CH; and R² is C₁₋₃ fluoroalkyl.

In some embodiments, Z is N. In some embodiments, Z is CH.

The present application further provides a compound of Formula (Ia):

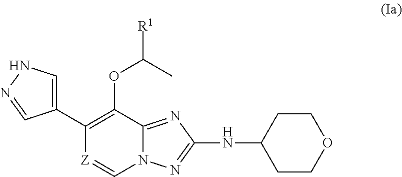

or a pharmaceutically acceptable salt thereof, wherein:
Z is CH or N; and
R¹ is selected from CH₃, CHF₂, and CF₃.

In some embodiments, Z is CH.

In some embodiments, Z is N.

In some embodiments, R¹ is selected from CH₃ and CHF₂.

In some embodiments, R¹ is selected from CH₃ and CF₃.

In some embodiments, R¹ is selected from CHF₂ and CF₃.

In some embodiments, R¹ is CH₃.

In some embodiments, R¹ is CHF₂.

In some embodiments, R¹ is CF₃.

In some embodiments, Z is CH; and R¹ is selected from CH₃, CHF₂, and CF₃.

In some embodiments, Z is N; and R¹ is selected from CH₃, CHF₂, and CF₃.

In some embodiments, Z is CH or N; and R¹ is selected from CH₃.

In some embodiments, Z is CH or N; and R¹ is selected from CHF₂.

In some embodiments, Z is CH or N; and R¹ is selected from CF₃.

In some embodiments, Z is CH and R¹ is selected from CH₃ and CHF₂.

In some embodiments, Z is CH and R¹ is selected from CH₃ and CF₃.

In some embodiments, Z is CH and R¹ is selected from CHF₂ and CF₃.

In some embodiments, Z is CH and R¹ is CH₃.

In some embodiments, Z is CH and R¹ is CHF₂.

In some embodiments, Z is CH and R¹ is CF₃.

In some embodiments, Z is N and R¹ is selected from CH₃ and CHF₂.

In some embodiments, Z is N and R¹ is selected from CH₃ and CF₃.

In some embodiments, Z is N and R¹ is selected from CHF₂ and CF₃.

In some embodiments, Z is N and R¹ is CH₃.

In some embodiments, Z is N and R¹ is CHF₂.

In some embodiments, Z is N and R¹ is CF₃.

In some embodiments, the compound is a compound having Formula (II):

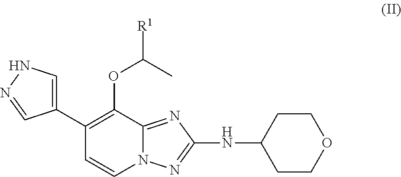

or a pharmaceutically acceptable salt thereof.

In some embodiments, the compound is a compound having Formula (III):

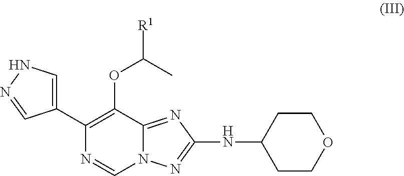

or a pharmaceutically acceptable salt thereof.

In some embodiments, 1, 2, 3, 4, 5, 6, 7, 8, 9, or 10 hydrogen atoms, attached to carbon atoms of the compounds, as described herein, are optionally replaced by deuterium atoms.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the term “Cₙ₋ₘ alkyl,” employed alone or in combination with other terms, refers to a saturated hydrocarbon group that may be straight-chain or branched, having n to m carbons. Examples of alkyl moieties include, but are not limited to, chemical groups such as methyl (Me), ethyl (Et), n-propyl (n-Pr), isopropyl (i-Pr), n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “halo” refers to F, Cl, Br, or I. In some embodiments, halo is F, Cl, or Br. In some embodiments, halo is F or Cl. In some embodiments, halo is F. In some embodiments, halo is Cl.

As used herein, the term “Cₙ₋ₘ fluoroalkyl” refers to an alkyl group having from one fluoro atom to 2s+1 fluoro atoms, where “s” is the number of carbon atoms in the alkyl group, wherein the alkyl group has n to m carbon atoms. In some embodiments, the alkyl group of the fluoroalkyl has 1 to 6, 1 to 4, or 1 to 3 carbon atoms. Example fluoroalkyl groups include CF₃, C₂F₅, CHF₂, CH₂F, and the like.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present disclosure are described and may be isolated as a mixture of isomers or as separated isomeric forms. In some embodiments, the compound has the (R)-configuration. In some embodiments, the compound has the (S)-configuration. The Formulas (e.g., Formula (I), (II), etc.) provided herein include stereoisomers of the compounds.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, 2-hydroxypyridine and 2-pyridone, and 1H- and 2H-pyrazole. Tautomeric forms can be in equilibrium or sterically locked into one form by appropriate substitution.

All compounds, and pharmaceutically acceptable salts thereof, can be found together with other substances such as water and solvents (e.g., hydrates and solvates) or can be isolated.

In some embodiments, preparation of compounds can involve the addition of acids or bases to affect, for example, catalysis of a desired reaction or formation of salt forms such as acid addition salts.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

As will be appreciated by those skilled in the art, the compounds provided herein, including salts and stereoisomers thereof, can be prepared using known organic synthesis techniques and can be synthesized according to any of numerous possible synthetic routes, such as those provided in the Schemes below.

The reactions for preparing compounds described herein can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

The expressions “ambient temperature” or “room temperature” or “r.t.” as used herein, are understood in the art, and refer generally to a temperature, e.g., a reaction temperature, that is about the temperature of the room in which the reaction is carried out, for example, a temperature from about 20° C. to about 30° C.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

The Schemes below provide general guidance in connection with preparing the compounds of the invention. One skilled in the art would understand that the preparations shown in the Schemes can be modified or optimized using general knowledge of organic chemistry to prepare various compounds of the invention.

Compounds of Formula (I) can be synthesized using a process shown in Scheme 1. Demethylation of appropriately substituted halides 1-1 with BBr₃ in an appropriate solvent (e.g., CH₂Cl₂) affords compounds of Formula 1-2. Alternatively, compounds of Formula 1-2 are commercially available. Nucleophilic substitution reactions of compounds of Formula 1-2 with appropriate electrophiles 1-3 under appropriate conditions (e.g., in the presence of a base, such as potassium carbonate or cesium carbonate, in an appropriate solvent, such as CH₃CN or DMF) affords compounds of Formula 1-4. Palladium-catalyzed cross-coupling reactions of aryl halides 1-4 and appropriately protected pyrazole boronic acids/esters 1-5 under appropriate conditions (e.g., in the presence of a palladium catalyst, such as chloro(2-dicyclohexylphosphino-2′,4′,6′-triisopropyl-1,1′-biphenyl)[2-(2′-amino-1,1′-biphenyl)]palladium(II) or [1,1′-bis(diphenylphosphino)ferrocene]dichloropalladium(II), complex with dichloromethane, and a base, such as K₃PO₄) afford compounds of Formula 1-6. Nucleophilic addition of compounds 1-6 to O-ethyl carbonisothiocyanatidate 1-7 in an appropriate solvent (e.g., CH₃CN, 1,4-dioxane) affords intermediate compounds 1-8. Cyclization of 1-8 with hydroxylamine hydrochloride/NN-diisopropylethylamine in an appropriate solvent (e.g., MeOH/EtOH) furnishes the aminobicyclic cores 1-9. Halogenation of compounds 1-9 (e.g., with an appropriate halogenating reagent such as copper(II) bromide or diiodomethane, and in the presence of tert-butyl nitrite in an appropriate solvent, such as CH₃CN) generates aryl halides 1-10. Transition metal catalyzed C—N bond forming reactions between the compounds 1-10 and tetrahydro-2H-pyran-4-amine under appropriate conditions (e.g., in the presence of a palladium catalyst, such as methanesulfonato[2-(di-1-adamantylphosphino)-3,6-dimethoxy-2′,4′,6′-tri-i-propyl-1,1′-biphenyl](2′-amino-1,1′-biphenyl-2-yl)palladium(II), and a base, such as sodium tert-butoxide, in an appropriate solvent, such as 1,4-dioxane or THF), followed by pyrazole deprotection under appropriate conditions (e.g., in the presence of an acid, such as 4 Molar HCl in 1,4-dioxane, in an appropriate solvent, such as MeOH) affords compounds of Formula I. Alternatively, pyrazole deprotection under appropriate conditions (e.g., 4 Molar HCl in 1,4-dioxane) in an appropriate solvent (e.g., MeOH/CH₂Cl₂) affords compounds 1-11. Reductive amination between the compounds 1-11 and tetrahydro-4H-pyran-4-one with an appropriate reducing agent (e.g., NaBH(OAc)₃) in an appropriate solvent (e.g., DMF/TFA) affords compounds of Formula I. Alternatively, reductive amination between the compounds 1-9 and tetrahydro-4H-pyran-4-one with an appropriate reducing agent (e.g., NaBH(OAc)₃) in an appropriate solvent (e.g., DMF/TFA) provides direct access to compounds of Formula I.

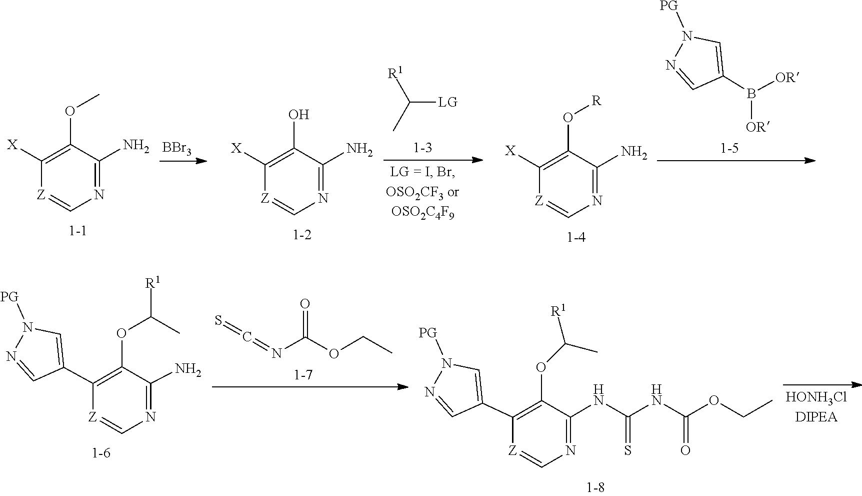

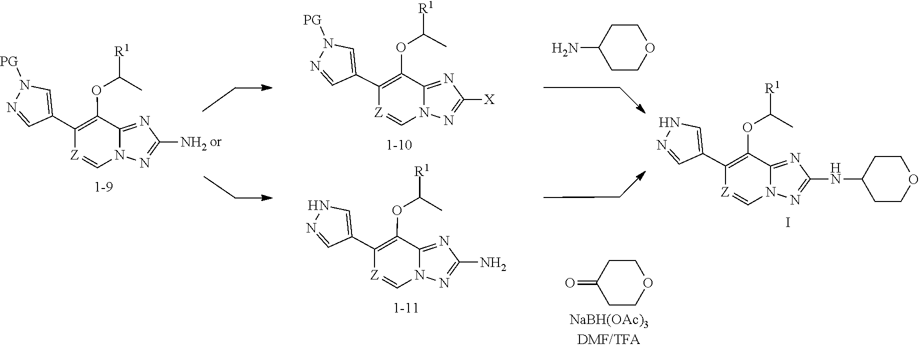

For the synthesis of particular compounds, the general schemes described above can be modified. For example, the products or intermediates can be modified to introduce particular functional groups. Alternatively, the substituents can be modified at any step of the overall synthesis by methods know to one skilled in the art, e.g., as described by Larock, Comprehensive Organic Transformations: A Guide to Functional Group Preparations (Wiley, 1999); and Katritzky et al. (Ed.), Comprehensive Organic Functional Group Transformations (Pergamon Press 1996).

Starting materials, reagents and intermediates whose synthesis is not described herein are either commercially available, known in the literature, or may be prepared by methods known to one skilled in the art.

It will be appreciated by one skilled in the art that the processes described are not the exclusive means by which compounds of the invention may be synthesized and that a broad repertoire of synthetic organic reactions is available to be potentially employed in synthesizing compounds of the invention. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H, ¹⁹F, or ¹³C), infrared spectroscopy, spectrophotometry (e.g., UV-visible), mass spectrometry or by chromatographic methods such as high performance liquid chromatography (HPLC) or thin layer chromatography (TLC).

### Methods of Use

Compounds of the present disclosure can inhibit CDK2 and therefore are useful for treating diseases wherein the underlying pathology is, wholly or partially, mediated by CDK2. Such diseases include cancer and other diseases with proliferation disorder. In some embodiments, the present disclosure provides treatment of an individual or a patient in vivo using a compound of Formula (I) or a salt thereof such that growth of cancerous tumors is inhibited. A compound of Formula (I) or of any of the formulas as described herein, or a compound as recited in any of the claims and described herein, or a salt thereof, can be used to inhibit the growth of cancerous tumors with aberrations that activate the CDK2 kinase activity. These include, but are not limited to, disease (e.g., cancers) that are characterized by amplification or overexpression of CCNE1 such as ovarian cancer, uterine carcinosarcoma and breast cancer and p27 inactivation such as breast cancer and melanomas. Accordingly, in some embodiments of the methods, the patient has been previously determined to have an amplification of the cyclin E1 (CCNE1) gene and/or an expression level of CCNE1 in a biological sample obtained from the human subject that is higher than a control expression level of CCNE1. Alternatively, a compound of Formula (I) or of any of the formulas as described herein, or a compound as recited in any of the claims and described herein, or a salt thereof, can be used in conjunction with other agents or standard cancer treatments, as described below. In one embodiment, the present disclosure provides a method for inhibiting growth of tumor cells in vitro. The method includes contacting the tumor cells in vitro with a compound of Formula (I) or of any of the formulas as described herein, or of a compound as recited in any of the claims and described herein, or of a salt thereof. In another embodiment, the present disclosure provides a method for inhibiting growth of tumor cells with CCNE1 amplification and overexpression in an individual or a patient. The method includes administering to the individual or patient in need thereof a therapeutically effective amount of a compound of Formula (I) or of any of the formulas as described herein, or of a compound as recited in any of the claims and described herein, or a salt or a stereoisomer thereof.

In some embodiments, provided herein is a method of inhibiting CDK2, comprising contacting the CDK2 with a compound of Formula (I) or any of the formulas as described herein, a compound as recited in any of the claims and described herein, or a salt thereof. In some embodiments, provided herein is a method of inhibiting CDK2 in a patient, comprising administering to the patient a compound of Formula (I) or any of the formulas as described herein, a compound as recited in any of the claims and described herein, or a salt thereof.

In some embodiments, provided herein is a method for treating cancer. The method includes administering to a patient (in need thereof), a therapeutically effective amount of a compound of Formula (I) or any of the formulas as described herein, a compound as recited in any of the claims and described herein, or a salt thereof. In another embodiment, the cancer is characterized by amplification or overexpression of CCNE1. In some embodiments, the cancer is ovarian cancer or breast cancer, characterized by amplification or overexpression of CCNE1.

In some embodiments, provided herein is a method of treating a disease or disorder associated with CDK2 in a patient, comprising administering to the patient a therapeutically effective amount of a compound of Formula (I) or any of the formulas as described herein, a compound as recited in any of the claims and described herein, or a salt thereof. In some embodiments, the disease or disorder associated with CDK2 is associated with an amplification of the cyclin E1 (CCNE1) gene and/or overexpression of CCNE1.

In some embodiments, the disease or disorder associated with CDK2 is N-myc amplified neuroblastoma cells (see Molenaar, et al., Proc Natl Acad Sci USA 106(31): 12968-12973) K-Ras mutant lung cancers (see Hu, S., et al., Mol Cancer Ther, 2015. 14(11): 2576-85, and cancers with FBW7 mutation and CCNE1 overexpression (see Takada, et al., Cancer Res, 2017. 77(18): 4881-4893).

The compounds of the present application have the ability to penetrate the blood brain barrier. Such penetrants are useful in the treatment of cancers that have metathesized to the brain. In some embodiments, the compound or the salt described herein is a blood brain barrier penetrant. The ability of a compound to penetrate the blood brain barrier can be determined by an appropriate assay or screening technique such as detailed in Example B. In some embodiments, the cancer is a cancer which has metathesized. In some embodiment, the metastatic cancer has metathesized to the brain. In some embodiments, the cancer is a metastatic cancer is metastatic breast cancer. In some embodiment, the metastatic breast cancer has metathesized to the brain. In some embodiments, the cancer is advanced breast cancer. In some embodiments, the metastatic cancer is metastatic lung cancer (i.e., non-small cell lung cancer or small cell lung cancer). In some embodiments, the cancer is advanced lung cancer (i.e., non-small cell lung cancer or small cell lung cancer). In some embodiment, the metastatic lung cancer has metathesized to the brain.

In some embodiments, the disease or disorder associated with CDK2 is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the disease or disorder associated with CDK2 is lung adenocarcinoma, breast invasive carcinoma, uterine carcinosarcoma, ovarian serous cystadenocarcinoma, or stomach adenocarcinoma.

In some embodiments, the disease or disorder associated with CDK2 is an adenocarcinoma, carcinoma, or cystadenocarcinoma.

In some embodiments, the disease or disorder associated with CDK2 is uterine cancer, ovarian cancer, stomach cancer, esophageal cancer, lung cancer, bladder cancer, pancreatic cancer, or breast cancer.

In some embodiments, the disease or disorder associated with CDK2 is a cancer.

In some embodiments, the cancer is characterized by amplification or overexpression of CCNE1. In some embodiments, the cancer is ovarian cancer or breast cancer, characterized by amplification or overexpression of CCNE1.

In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSI^{high}). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that compounds of Formula (I), or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual,” “patient,” and “subject,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

### I. Cancer Therapies

Cancer cell growth and survival can be impacted by dysfunction in multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

One or more additional pharmaceutical agents such as, for example, chemotherapeutics, anti-inflammatory agents, steroids, immunosuppressants, immune-oncology agents, metabolic enzyme inhibitors, chemokine receptor inhibitors, and phosphatase inhibitors, as well as targeted therapies such as Bcr-Abl, Flt-3, EGFR, HER2, JAK, c-MET, VEGFR, PDGFR, c-Kit, IGF-1R, RAF, FAK, and CDK4/6 kinase inhibitors such as, for example, those described in WO 2006/056399 can be used in combination with the compounds of the present disclosure for treatment of CDK2-associated diseases, disorders or conditions. Other agents such as therapeutic antibodies can be used in combination with the compounds of the present disclosure for treatment of CDK2-associated diseases, disorders or conditions. The one or more additional pharmaceutical agents can be administered to a patient simultaneously or sequentially.

In some embodiments, the CDK2 inhibitor is administered or used in combination with a BCL2 inhibitor or a CDK4/6 inhibitor.

The compounds as disclosed herein can be used in combination with one or more other enzyme/protein/receptor inhibitors therapies for the treatment of diseases, such as cancer and other diseases or disorders described herein. Examples of diseases and indications treatable with combination therapies include those as described herein. Examples of cancers include solid tumors and non-solid tumors, such as liquid tumors, and blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, BCL2, CDK4/6, TGF-βR, PKA, PKG, PKC, CaM-kinase, phosphorylase kinase, MEKK, ERK, MAPK, mTOR, EGFR, HER2, HER3, HER4, INS—R, IDH2, IGF-1R, IR—R, PDGFαR, PDGFβR, PI3K (alpha, beta, gamma, delta, and multiple or selective), CSF1R, KIT, FLK-II, KDR/FLK-1, FLK-4, fit-1, FGFR1, FGFR2, FGFR3, FGFR4, c-Met, PARP, Ron, Sea, TRKA, TRKB, TRKC, TAM kinases (Axl, Mer, Tyro3), FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., pemigatinib (INCB54828), INCB62079), an EGFR inhibitor (also known as ErB-1 or HER-1; e.g., erlotinib, gefitinib, vandetanib, orsimertinib, cetuximab, necitumumab, or panitumumab), a VEGFR inhibitor or pathway blocker (e.g. bevacizumab, pazopanib, sunitinib, sorafenib, axitinib, regorafenib, ponatinib, cabozantinib, vandetanib, ramucirumab, lenvatinib, ziv-aflibercept), a PARP inhibitor (e.g., olaparib, rucaparib, veliparib or niraparib), a JAK inhibitor (JAK1 and/or JAK2, e.g., ruxolitinib or baricitinib; JAK1, e.g., itacitinib (INCB39110), INCB052793, or INCB054707), an IDO inhibitor (e.g., epacadostat, NLG919, or BMS-986205, MK7162), an LSD1 inhibitor (e.g., GSK2979552, INCB59872 and INCB60003), a TDO inhibitor, a PI3K-delta inhibitor (e.g., parsaclisib (INCB50465) or INCB50797), a PI3K-gamma inhibitor such as PI3K-gamma selective inhibitor, a Pim inhibitor (e.g., INCB53914), a CSF1R inhibitor, a TAM receptor tyrosine kinases (Tyro-3, Axl, and Mer; e.g., INCB081776), an adenosine receptor antagonist (e.g., A2a/A2b receptor antagonist), an HPK1 inhibitor, a chemokine receptor inhibitor (e.g., CCR2 or CCR5 inhibitor), a SHP1/2 phosphatase inhibitor, a histone deacetylase inhibitor (HDAC) such as an HDAC8 inhibitor, an angiogenesis inhibitor, an interleukin receptor inhibitor, bromo and extra terminal family members inhibitors (for example, bromodomain inhibitors or BET inhibitors such as INCB54329 and INCB57643), c-MET inhibitors (e.g., capmatinib), an anti-CD19 antibody (e.g., tafasitamab), an ALK2 inhibitor (e.g., INCB00928); or combinations thereof.

In some embodiments, the compound or salt described herein is administered with a PI3Kδ inhibitor. In some embodiments, the compound or salt described herein is administered with a JAK inhibitor. In some embodiments, the compound or salt described herein is administered with a JAK1 or JAK2 inhibitor (e.g., baricitinib or ruxolitinib). In some embodiments, the compound or salt described herein is administered with a JAK1 inhibitor. In some embodiments, the compound or salt described herein is administered with a JAK1 inhibitor, which is selective over JAK2.

Example antibodies for use in combination therapy include, but are not limited to, trastuzumab (e.g., anti-HER2), ranibizumab (e.g., anti-VEGF-A), bevacizumab (AVASTIN™ e.g., anti-VEGF), panitumumab (e.g., anti-EGFR), cetuximab (e.g., anti-EGFR), rituxan (e.g., anti-CD20), and antibodies directed to c-MET.

One or more of the following agents may be used in combination with the compounds of the present disclosure and are presented as a non-limiting list: a cytostatic agent, cisplatin, doxorubicin, taxotere, taxol, etoposide, irinotecan, camptosar, topotecan, paclitaxel, docetaxel, epothilones, tamoxifen, 5-fluorouracil, methotrexate, temozolomide, cyclophosphamide, SCH 66336, R115777, L778,123, BMS 214662, IRESSA™(gefitinib), TARCEVA™ (erlotinib), antibodies to EGFR, intron, ara-C, adriamycin, cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, oxaliplatin, leucovirin, ELOXATIN™ (oxaliplatin), pentostatine, vinblastine, vincristine, vindesine, bleomycin, dactinomycin, daunorubicin, doxorubicin, epirubicin, idarubicin, mithramycin, deoxycoformycin, mitomycin-C, L-asparaginase, teniposide 17.alpha.-ethinylestradiol, diethylstilbestrol, testosterone, Prednisone, Fluoxymesterone, Dromostanolone propionate, testolactone, megestrolacetate, methylprednisolone, methyltestosterone, prednisolone, triamcinolone, chlorotrianisene, hydroxyprogesterone, aminoglutethimide, estramustine, medroxyprogesteroneacetate, leuprolide, flutamide, toremifene, goserelin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, avastin, HERCEPTIN™ (trastuzumab), BEXXAR™ (tositumomab), VELCADE™ (bortezomib), ZEVALIN™ (ibritumomab tiuxetan), TRISENOX™ (arsenic trioxide), XELODA™ (capecitabine), vinorelbine, porfimer, ERBITUX™ (cetuximab), thiotepa, altretamine, melphalan, trastuzumab, lerozole, fulvestrant, exemestane, ifosfomide, rituximab, C225 (cetuximab), Campath (alemtuzumab), clofarabine, cladribine, aphidicolon, rituxan, sunitinib, dasatinib, tezacitabine, Sml1, fludarabine, pentostatin, triapine, didox, trimidox, amidox, 3-AP, and MDL-101,731.

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, bispecific or multi-specific antibody, antibody drug conjugate, adoptive T cell transfer, Toll receptor agonists, RIG-I agonists, oncolytic virotherapy and immunomodulating small molecules, including thalidomide or JAK1/2 inhibitor, PI3Kδ inhibitor and the like. The compounds can be administered in combination with one or more anti-cancer drugs, such as a chemotherapeutic agent. Examples of chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat, and zoledronate.

Additional examples of chemotherapeutics include proteasome inhibitors (e.g., bortezomib), thalidomide, revlimid, and DNA-damaging agents such as melphalan, doxorubicin, cyclophosphamide, vincristine, etoposide, carmustine, and the like.

Example steroids include corticosteroids such as dexamethasone or prednisone.

Example Bcr-Abl inhibitors include imatinib mesylate (GLEEVAC™), nilotinib, dasatinib, bosutinib, and ponatinib, and pharmaceutically acceptable salts. Other example suitable Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491.

Example suitable Flt-3 inhibitors include midostaurin, lestaurtinib, linifanib, sunitinib, sunitinib, maleate, sorafenib, quizartinib, crenolanib, pacritinib, tandutinib, PLX3397 and ASP2215, and their pharmaceutically acceptable salts. Other example suitable Flt-3 inhibitors include compounds, and their pharmaceutically acceptable salts, as disclosed in WO 03/037347, WO 03/099771, and WO 04/046120.

Example suitable RAF inhibitors include dabrafenib, sorafenib, and vemurafenib, and their pharmaceutically acceptable salts. Other example suitable RAF inhibitors include compounds, and their pharmaceutically acceptable salts, as disclosed in WO 00/09495 and WO 05/028444.

Example suitable FAK inhibitors include VS-4718, VS-5095, VS-6062, VS-6063, BI853520, and GSK2256098, and their pharmaceutically acceptable salts. Other example suitable FAK inhibitors include compounds, and their pharmaceutically acceptable salts, as disclosed in WO 04/080980, WO 04/056786, WO 03/024967, WO 01/064655, WO 00/053595, and WO 01/014402.

Example suitable CDK4/6 inhibitors include palbociclib, ribociclib, trilaciclib, lerociclib, and abemaciclib, and their pharmaceutically acceptable salts. Other example suitable CDK4/6 inhibitors include compounds, and their pharmaceutically acceptable salts, as disclosed in WO 09/085185, WO 12/129344, WO 11/101409, WO 03/062236, WO 10/075074, and WO 12/061156.

In some embodiments, the compounds of the disclosure can be used in combination with one or more other kinase inhibitors including imatinib, particularly for treating patients resistant to imatinib or other kinase inhibitors.

In some embodiments, the compounds of the disclosure can be used in combination with a chemotherapeutic in the treatment of cancer, and may improve the treatment response as compared to the response to the chemotherapeutic agent alone, without exacerbation of its toxic effects. In some embodiments, the compounds of the disclosure can be used in combination with a chemotherapeutic provided herein. For example, additional pharmaceutical agents used in the treatment of multiple myeloma, can include, without limitation, melphalan, melphalan plus prednisone [MP], doxorubicin, dexamethasone, and Velcade (bortezomib). Further additional agents used in the treatment of multiple myeloma include Bcr-Abl, Flt-3, RAF and FAK kinase inhibitors. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM). Additive or synergistic effects are desirable outcomes of combining a CDK2 inhibitor of the present disclosure with an additional agent.

The agents can be combined with the present compound in a single or continuous dosage form, or the agents can be administered simultaneously or sequentially as separate dosage forms.

The compounds of the present disclosure can be used in combination with one or more other inhibitors or one or more therapies for the treatment of infections. Examples of infections include viral infections, bacterial infections, fungus infections or parasite infections.

In some embodiments, a corticosteroid such as dexamethasone is administered to a patient in combination with the compounds of the disclosure where the dexamethasone is administered intermittently as opposed to continuously.

The compounds of Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

In some further embodiments, combinations of the compounds of the disclosure with other therapeutic agents can be administered to a patient prior to, during, and/or after a bone marrow transplant or stem cell transplant. The compounds of the present disclosure can be used in combination with bone marrow transplant for the treatment of a variety of tumors of hematopoietic origin.

The compounds of Formula (I) or any of the formulas as described herein, a compound as recited in any of the claims and described herein, or salts thereof can be used in combination with vaccines, to stimulate the immune response to pathogens, toxins, and self-antigens. Examples of pathogens for which this therapeutic approach may be particularly useful include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas Aeruginosa.

Viruses causing infections treatable by methods of the present disclosure include, but are not limited to human papillomavirus, influenza, hepatitis A, B, C or D viruses, adenovirus, poxvirus, herpes simplex viruses, human cytomegalovirus, severe acute respiratory syndrome virus, Ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumps virus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

### II. Immune-Checkpoint Therapies

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CBL-B, CD20, CD28, CD40, CD70, CD122, CD96, CD73, CD47, CDK2, GITR, CSF1R, JAK, PI3K delta, PI3K gamma, TAM, arginase, HPK1, CD137 (also known as 4-1BB), ICOS, A2AR, B7-H3, B7-H4, BTLA, CTLA-4, LAG3, TIM3, TLR (TLR7/8), TIGIT, CD112R, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, TIGIT, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the compounds provided herein can be used in combination with one or more agonists of immune checkpoint molecules, e.g., OX40, CD27, GITR, and CD137 (also known as 4-1BB).

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1 or PD-L1, e.g., an anti-PD-1 or anti-PD-L1 monoclonal antibody. In some embodiments, the anti-PD-1 or anti-PD-L1 antibody is nivolumab, pembrolizumab, atezolizumab, durvalumab, avelumab, cemiplimab, atezolizumab, avelumab, tislelizumab, spartalizumab (PDR001), cetrelimab (JNJ-63723283), toripalimab (JS001), camrelizumab (SHR-1210), sintilimab (IBI308), AB122 (GLS-010), AMP-224, AMP-514/MEDI-0680, BMS936559, JTX-4014, BGB-108, SHR-1210, MEDI4736, FAZ053, BCD-100, KN035, CS1001, BAT1306, LZM009, AK105, HLX10, SHR-1316, CBT-502 (TQB2450), A167 (KL-A167), STI-A101 (ZKAB001), CK-301, BGB-A333, MSB-2311, HLX20, TSR-042, or LY3300054. In some embodiments, the inhibitor of PD-1 or PD-L1 is one disclosed in U.S. Pat. Nos. 7,488,802, 7,943,743, 8,008,449, 8,168,757, 8,217, 149, WO 03042402, WO 2008156712, WO 2010089411, WO 2010036959, WO 2011066342, WO 2011159877, WO 2011082400, or WO 2011161699, which are each incorporated herein by reference in its entirety.

In some embodiments, the antibody is an anti-PD-1 antibody, e.g., an anti-PD-1 monoclonal antibody. In some embodiments, the anti-PD-1 antibody is nivolumab, pembrolizumab, cemiplimab, spartalizumab, camrelizumab, cetrelimab, toripalimab, sintilimab, AB122, AMP-224, JTX-4014, BGB-108, BCD-100, BAT1306, LZM009, AK105, HLX10, or TSR-042. In some embodiments, the anti-PD-1 antibody is nivolumab, pembrolizumab, cemiplimab, spartalizumab, camrelizumab, cetrelimab, toripalimab, or sintilimab. In some embodiments, the anti-PD-1 antibody is pembrolizumab. In some embodiments, the anti-PD-1 antibody is nivolumab. In some embodiments, the anti-PD-1 antibody is cemiplimab. In some embodiments, the anti-PD-1 antibody is spartalizumab. In some embodiments, the anti-PD-1 antibody is camrelizumab. In some embodiments, the anti-PD-1 antibody is cetrelimab. In some embodiments, the anti-PD-1 antibody is toripalimab. In some embodiments, the anti-PD-1 antibody is sintilimab. In some embodiments, the anti-PD-1 antibody is AB122. In some embodiments, the anti-PD-1 antibody is AMP-224. In some embodiments, the anti-PD-1 antibody is JTX-4014. In some embodiments, the anti-PD-1 antibody is BGB-108. In some embodiments, the anti-PD-1 antibody is BCD-100. In some embodiments, the anti-PD-1 antibody is BAT1306. In some embodiments, the anti-PD-1 antibody is LZM009. In some embodiments, the anti-PD-1 antibody is AK105. In some embodiments, the anti-PD-1 antibody is HLX10. In some embodiments, the anti-PD-1 antibody is TSR-042. In some embodiments, the anti-PD-1 monoclonal antibody is nivolumab or pembrolizumab. In some embodiments, the anti-PD-1 monoclonal antibody is MGA012. In some embodiments, the anti-PD1 antibody is SHR-1210. Other anti-cancer agent(s) include antibody therapeutics such as 4-1BB (e.g., urelumab, utomilumab). In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, avelumab, durvalumab, tislelizumab, BMS-935559, MEDI4736, atezolizumab (MPDL3280A;also known as RG7446), avelumab (MSB0010718C), FAZ053, KN035, CS1001, SHR-1316, CBT-502, A167, STI-A101, CK-301, BGB-A333, MSB-2311, HLX20, or LY3300054. In some embodiments, the anti-PD-L1 antibody is atezolizumab, avelumab, durvalumab, or tislelizumab. In some embodiments, the anti-PD-L1 antibody is atezolizumab. In some embodiments, the anti-PD-L1 antibody is avelumab. In some embodiments, the anti-PD-L1 antibody is durvalumab. In some embodiments, the anti-PD-L1 antibody is tislelizumab. In some embodiments, the anti-PD-L1 antibody is BMS-935559. In some embodiments, the anti-PD-L1 antibody is MEDI4736. In some embodiments, the anti-PD-L1 antibody is FAZ053. In some embodiments, the anti-PD-L1 antibody is KN035. In some embodiments, the anti-PD-L1 antibody is CS1001. In some embodiments, the anti-PD-L1 antibody is SHR-1316. In some embodiments, the anti-PD-L1 antibody is CBT-502. In some embodiments, the anti-PD-L1 antibody is A167. In some embodiments, the anti-PD-L1 antibody is STI-A101. In some embodiments, the anti-PD-L1 antibody is CK-301. In some embodiments, the anti-PD-L1 antibody is BGB-A333. In some embodiments, the anti-PD-L1 antibody is MSB-2311. In some embodiments, the anti-PD-L1 antibody is HLX20. In some embodiments, the anti-PD-L1 antibody is LY3300054.

In some embodiments, the inhibitor of an immune checkpoint molecule is a small molecule that binds to PD-L1, or a pharmaceutically acceptable salt thereof. In some embodiments, the inhibitor of an immune checkpoint molecule is a small molecule that binds to and internalizes PD-L1, or a pharmaceutically acceptable salt thereof. In some embodiments, the inhibitor of an immune checkpoint molecule is a compound selected from those in US 2018/0179201, US 2018/0179197, US 2018/0179179, US 2018/0179202, US 2018/0177784, US 2018/0177870, U.S. Ser. No. 16/369,654 (filed Mar. 29, 2019), and U.S. Ser. No. 62/688,164, or a pharmaceutically acceptable salt thereof, each of which is incorporated herein by reference in its entirety.

In some embodiments, the inhibitor of an immune checkpoint molecule is an inhibitor of KIR, TIGIT, LAIR1, CD160, 2B4 and TGFR beta.

In some embodiments, the inhibitor is MCLA-145.

In some embodiments, the inhibitor of an immune checkpoint molecule is an inhibitor of CTLA-4, e.g., an anti-CTLA-4 antibody. In some embodiments, the anti-CTLA-4 antibody is ipilimumab, tremelimumab, AGEN1884, or CP-675,206.

In some embodiments, the inhibitor of an immune checkpoint molecule is an inhibitor of LAG3, e.g., an anti-LAG3 antibody. In some embodiments, the anti-LAG3 antibody is BMS-986016, LAG525, INCAGN2385, or eftilagimod alpha (IMP321).

In some embodiments, the inhibitor of an immune checkpoint molecule is an inhibitor of CD73. In some embodiments, the inhibitor of CD73 is oleclumab.

In some embodiments, the inhibitor of an immune checkpoint molecule is an inhibitor of TIGIT. In some embodiments, the inhibitor of TIGIT is OMP-31M32.

In some embodiments, the inhibitor of an immune checkpoint molecule is an inhibitor of VISTA. In some embodiments, the inhibitor of VISTA is JNJ-61610588 or CA-170.

In some embodiments, the inhibitor of an immune checkpoint molecule is an inhibitor of B7-H3. In some embodiments, the inhibitor of B7-H3 is enoblituzumab, MGD009, or 8119.

In some embodiments, the inhibitor of an immune checkpoint molecule is an inhibitor of KIR. In some embodiments, the inhibitor of KIR is lirilumab or IPH4102.

In some embodiments, the inhibitor of an immune checkpoint molecule is an inhibitor of A2aR. In some embodiments, the inhibitor of A2aR is CPI-444.

In some embodiments, the inhibitor of an immune checkpoint molecule is an inhibitor of TGF-beta. In some embodiments, the inhibitor of TGF-beta is trabedersen, galusertinib, or M7824.

In some embodiments, the inhibitor of an immune checkpoint molecule is an inhibitor of PI3K-gamma. In some embodiments, the inhibitor of PI3K-gamma is IPI-549.

In some embodiments, the inhibitor of an immune checkpoint molecule is an inhibitor of CD47. In some embodiments, the inhibitor of CD47 is Hu5F9-G4 or TTI-621.

In some embodiments, the inhibitor of an immune checkpoint molecule is an inhibitor of CD73. In some embodiments, the inhibitor of CD73 is MEDI9447.

In some embodiments, the inhibitor of an immune checkpoint molecule is an inhibitor of CD70. In some embodiments, the inhibitor of CD70 is cusatuzumab or BMS-936561.

In some embodiments, the inhibitor of an immune checkpoint molecule is an inhibitor of TIM3, e.g., an anti-TIM3 antibody. In some embodiments, the anti-TIM3 antibody is INCAGN2390, MBG453, or TSR-022.

In some embodiments, the inhibitor of an immune checkpoint molecule is an inhibitor of CD20, e.g., an anti-CD20 antibody. In some embodiments, the anti-CD20 antibody is obinutuzumab or rituximab.

In some embodiments, the agonist of an immune checkpoint molecule is an agonist of OX40, CD27, CD28, GITR, ICOS, CD40, TLR7/8, and CD137 (also known as 4-1BB).

In some embodiments, the agonist of CD137 is urelumab. In some embodiments, the agonist of CD137 is utomilumab.

In some embodiments, the agonist of an immune checkpoint molecule is an inhibitor of GITR. In some embodiments, the agonist of GITR is TRX518, MK-4166, INCAGN1876, MK-1248, AMG228, BMS-986156, GWN323, MEDI1873, or MEDI6469.In some embodiments, the agonist of an immune checkpoint molecule is an agonist of OX40, e.g., OX40 agonist antibody or OX40L fusion protein. In some embodiments, the anti-OX40 antibody is INCAGN01949, MEDI0562 (tavolimab), MOXR-0916, PF-04518600, GSK3174998, BMS-986178, or 9B12.. In some embodiments, the OX40L fusion protein is MEDI6383.

In some embodiments, the agonist of an immune checkpoint molecule is an agonist of CD40. In some embodiments, the agonist of CD40 is CP-870893, ADC-1013, CDX-1140, SEA-CD40, R07009789, JNJ-64457107, APX-005M, or Chi Lob 7/4.

In some embodiments, the agonist of an immune checkpoint molecule is an agonist of ICOS. In some embodiments, the agonist of ICOS is GSK-3359609, JTX-2011, or MEDI-570.

In some embodiments, the agonist of an immune checkpoint molecule is an agonist of CD28. In some embodiments, the agonist of CD28 is theralizumab.

In some embodiments, the agonist of an immune checkpoint molecule is an agonist of CD27. In some embodiments, the agonist of CD27 is varlilumab.

In some embodiments, the agonist of an immune checkpoint molecule is an agonist of TLR7/8. In some embodiments, the agonist of TLR7/8 is MEDI9197.

The compounds of the present disclosure can be used in combination with bispecific antibodies. In some embodiments, one of the domains of the bispecific antibody targets PD-1, PD-L1, CTLA-4, GITR, OX40, TIM3, LAG3, CD137, ICOS, CD3 or TGFβ receptor. In some embodiments, the bispecific antibody binds to PD-1 and PD-L1. In some embodiments, the bispecific antibody that binds to PD-1 and PD-L1 is MCLA-136. In some embodiments, the bispecific antibody binds to PD-L1 and CTLA-4. In some embodiments, the bispecific antibody that binds to PD-L1 and CTLA-4 is AK104.

In some embodiments, the compounds of the disclosure can be used in combination with one or more metabolic enzyme inhibitors. In some embodiments, the metabolic enzyme inhibitor is an inhibitor of IDO1, TDO, or arginase. Examples of IDO1 inhibitors include epacadostat, NLG919, BMS-986205, PF-06840003, IOM2983, RG-70099 and LY338196.

As provided throughout, the additional compounds, inhibitors, agents, etc. can be combined with the present compound in a single or continuous dosage form, or they can be administered simultaneously or sequentially as separate dosage forms.

### Pharmaceutical Formulations and Dosage Forms

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the disclosure or a pharmaceutically acceptable salt thereof, in combination with one or more pharmaceutically acceptable carriers (excipients). In some embodiments, the composition is suitable for topical administration. In making the compositions of the disclosure, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form, each dosage containing from about 5 to about 1000 mg (1 g), or more, such as about 100 to about 500 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions of the disclosure contain from about 5 to about 50 mg of the active ingredient. One having ordinary skill in the art will appreciate that this embodies compositions containing about 5 to about 10, about 10 to about 15, about 15 to about 20, about 20 to about 25, about 25 to about 30, about 30 to about 35, about 35 to about 40, about 40 to about 45, or about 45 to about 50 mg of the active ingredient.

In some embodiments, the compositions of the disclosure contain from about 50 to about 500 mg of the active ingredient. One having ordinary skill in the art will appreciate that this embodies compositions containing about 50 to about 100, about 100 to about 150, about 150 to about 200, about 200 to about 250, about 250 to about 300, about 350 to about 400, or about 450 to about 500 mg of the active ingredient.

In some embodiments, the compositions of the disclosure contain from about 500 to about 1000 mg of the active ingredient. One having ordinary skill in the art will appreciate that this embodies compositions containing about 500 to about 550, about 550 to about 600, about 600 to about 650, about 650 to about 700, about 700 to about 750, about 750 to about 800, about 800 to about 850, about 850 to about 900, about 900 to about 950, or about 950 to about 1000 mg of the active ingredient.

Similar dosages may be used of the compounds described herein in the methods and uses of the disclosure.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the disclosure can further include one or more additional pharmaceutical agents such as a chemotherapeutic, steroid, anti-inflammatory compound, or immunosuppressant, examples of which are listed herein.

### Labeled Compounds and Assay Methods

Another aspect of the present disclosure relates to labeled compounds of the disclosure (radio-labeled, fluorescent-labeled, etc.) that would be useful not only in imaging techniques but also in assays, both in vitro and in vivo, for localizing and quantitating CDK2 in tissue samples, including human, and for identifying CDK2 activators by inhibition binding of a labeled compound. Substitution of one or more of the atoms of the compounds of the present disclosure can also be useful in generating differentiated ADME (Adsorption, Distribution, Metabolism and Excretion). Accordingly, the present disclosure includes CDK2 assays that contain such labeled or substituted compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms (e.g., one or more hydrogen atoms of a C₁₋₆ alkyl group of Formula (I) can be optionally substituted with deuterium atoms, such as —CD₃ being substituted for —CH₃). In some embodiments, alkyl groups of the disclosed Formulas (e.g., Formula (I)) can be perdeuterated.

One or more constituent atoms of the compounds presented herein can be replaced or substituted with isotopes of the atoms in natural or non-natural abundance. In some embodiments, the compound includes at least one deuterium atom. For example, one or more hydrogen atoms in a compound presented herein can be replaced or substituted by deuterium (e.g., one or more hydrogen atoms of a C₁₋₆ alkyl group can be replaced by deuterium atoms, such as —CD₃ being substituted for —CH₃). In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

In some embodiments, 1, 2, 3, 4, 5, 6, 7, or 8 hydrogen atoms, attached to carbon atoms of alkyl, alkenyl, alkynyl, aryl, phenyl, cycloalkyl, heterocycloalkyl, or heteroaryl substituents or —C₁₋₄ alkyl-, alkylene, alkenylene and alkynylene linking groups, as described herein, are optionally replaced by deuterium atoms.

Synthetic methods for including isotopes into organic compounds are known in the art (Deuterium Labeling in Organic Chemistry by Alan F. Thomas, New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et al. J. Med. Chem. 2011, 54, 201-210; R. Xu et al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro CDK2 labeling and competition assays, compounds that incorporate ³H, ¹⁴C, ⁸²Br, ¹²⁵I, ¹³¹I, or ³⁵S can be useful. For radio-imaging applications ¹¹C, ¹⁸F, ¹²⁵I, ¹²³I, ¹²⁴I, ¹³¹I, ⁷⁵Br, ⁷⁶Br, or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S, and ⁸²Br.

The present disclosure can further include synthetic methods for incorporating radio-isotopes into compounds of the disclosure. Synthetic methods for incorporating radio-isotopes into organic compounds are well known in the art, and one of ordinary skill in the art will readily recognize the methods applicable for the compounds of disclosure.

A labeled compound of the disclosure can be used in a screening assay to identify/evaluate compounds. For example, a newly synthesized or identified compound (i.e., test compound) which is labeled can be evaluated for its ability to bind and activate CDK2 by monitoring its concentration variation when contacting with CDK2, through tracking of the labeling. For example, a test compound (labeled) can be evaluated for its ability to reduce binding of another compound which is known to inhibit CDK2 (i.e., standard compound). Accordingly, the ability of a test compound to compete with the standard compound for binding to CDK2 directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### Kits

The present disclosure also includes pharmaceutical kits useful, for example, in the treatment or prevention of CDK2-associated diseases or disorders (such as, e.g., cancer, an inflammatory disease, a cardiovascular disease, or a neurodegenerative disease) which include one or more containers containing a pharmaceutical composition comprising a therapeutically effective amount of a compound of the disclosure. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Biomarkers and Pharmacodynamics Markers

The disclosure further provides predictive markers (e.g., biomarkers and pharmacodynamic markers, e.g., gene copy number, gene sequence, expression levels, or phosphorylation levels) to identify those human subjects having, suspected of having, or at risk of developing a disease or disorder associated with CDK2 for whom administering a CDK2 inhibitor (“a CDK2 inhibitor” as used herein refers to a compound of the disclosure, or a pharmaceutically acceptable salt thereof) is likely to be effective. The disclosure also provides pharmacodynamic markers (e.g., phosphorylation levels) to identify those human subjects having, suspected of having, or at risk of developing a disease or disorder associated with CDK2 whom are responding to a CDK2 inhibitor. The use of CCNE1, p16, and Rb S780 is further described in U.S. Patent Publ. No. 2020/0316064), the figures and disclosure of which is incorporated by reference herein in its entirety.

The methods are based, at least in part, on the discovery that the functional status of cyclin dependent kinase inhibitor 2A (“CDKN2A”; also referred to as “p16”) is a biomarker for predicting sensitivity to CDK2-targeting therapies in G1/S-specific cyclin-E1-(“CCNE1-”) amplified cells suitable for use in patient stratification. In addition, the present invention is based, at least in part, on the discovery that, in CCNE1-amplified cell lines, the level of human retinoblastoma associated protein (“Rb”) phosphorylation at the serine corresponding to amino acid position 780 of SEQ ID NO:3 is a pharmacodynamic marker for CDK2 activity and is suitable for use in measuring CDK2 enzymatic activity in cellular assay or preclinical and clinical applications, such as, e.g., monitoring the progress of or responsiveness to treatment with a CDK2 inhibitor.

### CCNE1 and p16

CCNE1 and p16 have been identified in the Examples as genes, in combination, useful in predicting responsiveness (e.g., improvement in disease as evidenced by disease remission/resolution) of a subject having a disease or disorder associated with CDK2 to a CDK2 inhibitor.

p16 (also known as cyclin-dependent kinase inhibitor 2A, cyclin-dependent kinase 4 inhibitor A, multiple tumor suppressor 1, and p16-INK4a) acts as a negative regulator of the proliferation of normal cells by interacting with CDK4 and CDK6. p16 is encoded by the cyclin dependent kinase inhibitor 2A (“CDKN2A”) gene (GenBank Accession No. NM_000077). The cytogenic location of the CDKN2A gene is 9p21.3, which is the short (p) arm of chromosome 9 at position 21.3. The molecular location of the CDKN2A gene is base pairs 21,967,752 to 21,995,043 on chromosome 9 (Homo sapiens Annotation Release 109, GRCh38.p12). Genetic and epigenetic abnormalities in the gene encoding p16 are believed to lead to escape from senescence and cancer formation (Okamoto et al., 1994, PNAS 91(23):11045-9). Nonlimiting examples of genetic abnormalities in the gene encoding p16 are described in Table A, below. The amino acid sequence of human p16 is provided below (GenBank Accession No. NP_000068/UniProtKB Accession No. P42771):

| (SEQ ID NO: 1) | |
|---|---|
| 1 | MEPAAGSSME PSADWLATAA ARGRVEEVRA LLEAGALPNA PNSYGRRPIQ VMMMGSARVA |
| 61 | ELLLLHGAEP NCADPATLTR PVHDAAREGF LDTLVVLHRA GARLDVRDAW GRLPVDLAEE |
| 121 | LGHRDVARYL RAAAGGTRGS NHARIDAAEG PSDIPD; |

CCNE1 is a cell cycle factor essential for the control of the cell cycle at the G1/S transition (Ohtsubo et al., 1995, Mol. Cell. Biol. 15:2612-2624). CCNE1 acts as a regulatory subunit of CDK2, interacting with CDK2 to form a serine/threonine kinase holoenzyme complex. The CCNE1 subunit of this holoenzyme complex provides the substrate specificity of the complex (Honda et al., 2005, EMBO 24:452-463). CCNE1 is encoded by the cycin E1 (“CCNE1”) gene (GenBank Accession No. NM_001238). The amino acid sequence of human CCNE1 is provided below (GenBank Accession No. NP_001229/UniProtKB Accession No. P24864):

| (SEQ ID NO: 2) | |
|---|---|
| 1 | mprerrerda kerdtmkedg gaefsarsrk rkanvtvflq dpdeemakid rtardqcgsq |
| 61 | pwdnnavcad pcsliptpdk edddrvypns tckpriiaps rgsplpvlsw anreevwkim |
| 121 | lnkektylrd qhfleqhpll qpkmrailld wlmevcevyk lhretfylaq dffdrymatq |
| 181 | envvktllql igisslfiaa kleeiyppkl hqfayvtdga csgdeiltme lmimkalkwr |
| 241 | lspltivswl nvymqvayln dlhevllpqy pqqifigiae lldlcvldvd clefpygila |
| 301 | asalyhfsss elmqkvsgyq wcdiencvkw mvpfamvire tgssklkhfr gvadedahni |
| 361 | qthrdsldll dkarakkaml seqnrasplp sglltppqsg kkqssgpema. |

The Examples demonstrate CDK2-knockdown inhibits proliferation of CCNE1-amplified cell lines, but not of CCNE1-non-amplified cell lines. Conversely, the Examples show that CDK4/6 inhibition inhibits proliferation of CCNE1-non-amplified cell lines, but not of CCNE1-amplified cell lines. The Examples further demonstrate that presence of a normal (e.g., non-mutated or non-deleted) p16 gene is required for the observed inhibition of cell proliferation in CCNE1-amplified cells treated with a CDK2-inhibitor. Accordingly, CCNE1 and p16 are, together, a combination biomarker: cells that respond to treatment with a CDK2 inhibitor display an amplification of the CCNE1 gene and/or an expression level of CCNE1 that is higher than a control expression level of CCNE1, and have a nucleotide sequence (e.g., a gene or an mRNA) that encodes the p16 protein (e.g., a p16 protein comprising the amino acid sequence of SEQ ID NO:1) and/or have p16 protein present, while control cells that do not respond to treatment with a CDK2 inhibitor do not have an amplification of the CCNE1 gene and/or an expression level of CCNE1 that is higher than a control expression level of CCNE1, and tend to have a mutated or deleted gene that encodes the p16 protein and/or lack expression of p16 protein.

Thus, the disclosure provides a method of treating a human subject having, suspected of having, or at risk of developing a disease or disorder associated with CDK2, comprising administering to the human subject a CDK2 inhibitor, wherein the human subject has been previously determined to: (i) (a) have a nucleotide sequence encoding a p16 protein comprising the amino acid sequence of SEQ ID NO:1, (b) have a CDKN2A gene lacking one or more inactivating nucleic acid substitutions and/or deletions, and/or (c) express a p16 protein, and (ii) (a) have an amplification of the CCNE1 gene and/or (b) have an expression level of CCNE1 in a biological sample obtained from the human subject that is higher than a control expression level of CCNE1. In certain embodiments, the predictive methods described herein predict that the subject will respond to treatment with the CDK2 inhibitor with at least 50%, at least 60%, at least 70%, at least 80%, at least 90%, at least 95%, at least 98% or 100% accuracy. For example, in some embodiments, if the predictive methods described herein are applied to 10 subjects having, suspected of having, or at risk of developing a disease or disorder associated with CDK2, and 8 of those 10 subjects are predicted to respond to treatment with a CDK2 inhibitor based on a predictive method described herein, and 7 of those 8 subjects do indeed respond to treatment with a CDK2 inhibitor, then the predictive method has an accuracy of 87.5% (7 divided by 8). A subject is considered to respond to the CDK2 inhibitor if the subject shows any improvement in disease status as evidenced by, e.g., reduction or alleviation in symptoms, disease remission/resolution, etc.

In some embodiments, the subject has a disease or disorder associated with CDK2. In some embodiments, the human subject has been previously determined to: (i) (a) have a nucleotide sequence encoding a p16 protein comprising the amino acid sequence of SEQ ID NO:1 and/or (b) a CDKN2A gene lacking one or more inactivating nucleic acid substitutions and/or deletions, and (ii) have an amplification of the CCNE1 gene in a biological sample obtained from the human subject. In some embodiments, the CDKN2A gene encodes a protein comprising the amino acid sequence of SEQ TD NO: 1. In specific embodiments, the CDKN2A gene encodes a protein comprising the amino acid sequence of SEQ ID NO: 1.

In specific embodiments, the one or more inactivating nucleic acid substitutions and/or deletions in the CDKN2A gene is as described in Table A. In specific embodiments, the one or more inactivating nucleic acid substitutions and/or deletions in the CDKN2A gene is as described in Yarbrough et al., Journal of the National Cancer Institute, 91(18):1569-1574, 1999; Liggett and Sidransky, Biology of Neoplasia, Journal of Oncology, 16(3):1197-1206, 1998, and Cairns et al., Nature Genetics, 11:210-212, 1995, each of which is incorporated by reference herein in its entirety.

TABLE A
CDKN2A gene substitutions, deletions, and modifications

| Description | Reference(s) |
|---|---|
| C to T transition converting codon 232 of the | RefSNP Accession No. rs121913388; |
| CDKN2A gene from an arginine codon to a stop | Kamb et al., Science 264: 436-440, |
| codon | 1994 |
| 19-basepair germline deletion at nucleotide 225 | RefSNP Accession No. rs587776716; |
| causing a reading-frame shift predicted to severely | Gruis et al., Nature Genet. 10: 351-353, |
| truncate p16 protein | 1995 |
| 6-basepair deletion at nucleotides 363-368 of the | Clin Var Accession No. |
| CDKN2A gene | RCV000010017.2; Liu et al., Oncogene |
| | 11: 405-412, 1995 |
| Mutation at chromosome 9: 21971058 predicted to | RefSNP Accession No. rs104894094; |
| substitute glycine corresponding to amino acid | Ciotti et al., Am. J. Hum. Genet. 67: |
| position 101 of SEQ ID NO: 1 with a tryptophan | 311-319, 2000 |
| Germline mutation constituting an in-frame 3- | Clin Var Accession No. |
| basepair duplication at nucleotide 332 in exon 2 of | RCV000010020.3; Borg et al., Cancer |
| the CDKN2A gene | Res. 56: 2497-2500, 1996 |
| Mutation predicted to substitute methionine | RefSNP Accession No. rs104894095; |
| corresponding to amino acid position 53 of SEQ ID | Harland et al., Hum. Molec. Genet. 6: |
| NO: 1 with an isoleucine | 2061-2067, 1997 |
| Mutation predicted to substitute arginine | RefSNP Accession No. rs104894097; |
| corresponding to amino acid position 24 of SEQ ID | Monzon et al., New Eng. J. Med. 338: |
| NO: 1 with a proline | 879-887, 1998 |
| 24-basepair repeat inserted at chromosome 9 | RefSNP Accession No. rs587780668; |
| between 21974795 and 21974796 (forward strand) | Pollock et al., Hum. Mutat. 11: 424- |
| | 431, 1998) |
| G-to-T transversion at nucleotide −34 of the | Clin Var Accession No. |
| CDKN2A gene | RCV000010024.5; Liu et al., Nature |
| | Genet. 21: 128-132, 1999 |
| Deletion of the p14(ARF)-specific exon 1-beta of | Clin Var Accession No. |
| CDKN2A | RCV000010026.2; Randerson-Moor et |
| | al., Hum. Molec. Genet. 10: 55-62, |
| | 2001 |
| Mutation predicted to substitute valine | RefSNP Accession No. rs104894098; |
| corresponding to amino acid position 126 of SEQ | Goldstein et al., Brit. J. Cancer 85: 527- |
| ID NO: 1 with an isoleucine | 530, 2001 |
| Transition (IVS2-105 A-G) in intron 2 of the | Clin Var Accession No. |
| CDKN2A gene creating a false GT splice donor site | RCV000010028.3; Harland et al., Hum. |
| 105 bases 5-prime of exon 3 resulting in aberrant | Molec. Genet. 10: 2679-2686, 2001 |
| splicing of the mRNA | |
| Mutation predicted to result in substitution of | RefSNP Accession No. rs113798404; |
| glycine corresponding to amino acid position 122 of | Hewitt et al., Hum. Molec. Genet. 11: |
| SEQ ID NO: 1 with an arginine | 1273-1279, 2002 |
| Mutation predicted to result in substitution of valine | RefSNP Accession No. rs113798404; |
| corresponding to amino acid position 59 of SEQ ID | Yakobson et al., Melanoma Res. 11: |
| NO: 1 with an arginine | 569-570, 2001 |
| Tandem germline339G-C transversion and a 340C- | RefSNP Accession Nos. rs113798404 |
| T transition in the CDKN2A gene resulting in | and rs104894104; Kannengiesser et al., |
| substitution of proline corresponding to amino acid | Genes Chromosomes Cancer 46: 751- |
| position 114 of SEQ ID NO: 1 with a serine | 760, 2007 |
| Mutation predicted to result in substitution of serine | RefSNP Accession No. rs104894109; |
| corresponding to amino acid position 56 of SEQ ID | Kannengiesser et al., Genes |
| NO: 1 with an isoleucine | Chromosomes Cancer 46: 751-760, |
| | 2007 |
| Mutation predicted to result in substitution of | RefSNP Accession No. rs137854599; |
| glycine corresponding to amino acid position 89 of | Goldstein et al., J. Med. Genet. 45: 284- |
| SEQ ID NO: 1 with an aspartic acid | 289, 2008 |
| Heterozygous A-to-G transition in exon 1B of the | Clin Var Accession no. |
| CDKN2A gene, affecting splicing of the p14(ARF) | RCV000022943.3; Binni et al., Clin. |
| isoform | Genet. 77: 581-586, 2010 |
| Heterozygous 5-bp duplication (19_23dup) in the | ClinVar Accession No. |
| CDKN2A gene, resulting in a frameshift and | RCV000030680.6; Harinck, F., Kluijt et |
| premature termination | al., J. Med. Genet. 49: 362-365, 2012 |
| Mutation predicted to result in substitution of | Yarbrough et al., Journal of the |
| aspartic acid corresponding to amino acid position | National Cancer Institute, 91(18): 1569- |
| 84 of SEQ ID NO: 1 with a valine | 1574 |
| Mutation predicted to result in substitution of | Yarbrough et al., Journal of the |
| aspartic acid corresponding to amino acid position | National Cancer Institute, 91(18): 1569- |
| 84 of SEQ ID NO: 1 with a glycine | 1574 |
| Mutation predicted to result in substitution of | Yarbrough et al., Journal of the |
| arginine corresponding to amino acid position 87 of | National Cancer Institute, 91(18): 1569- |
| SEQ ID NO: 1 with a proline | 1574 |
| Mutation predicted to result in substitution of | Yarbrough et al., Journal of the |
| proline corresponding to amino acid position 48 of | National Cancer Institute, 91(18): 1569- |
| SEQ ID NO: 1 with a leucine | 1574 |
| Mutation predicted to result in substitution of | Yarbrough et al., Journal of the |
| aspartic acid corresponding to amino acid position | National Cancer Institute, 91(18): 1569- |
| 74 of SEQ ID NO: 1 with a asparagine | 1574 |
| Mutation predicted to result in substitution of | Yarbrough et al., Journal of the |
| arginine corresponding to amino acid position 87 of | National Cancer Institute, 91(18): 1569- |
| SEQ ID NO: 1 with a leucine | 1574 |
| Mutation predicted to result in substitution of | Yarbrough et al., Journal of the |
| asparagine corresponding to amino acid position 71 | National Cancer Institute, 91(18): 1569- |
| of SEQ ID NO: 1 with a serine | 1574 |
| Mutation predicted to result in substitution of | Yarbrough et al., Journal of the |
| arginine corresponding to amino acid position 80 of | National Cancer Institute, 91(18): 1569- |
| SEQ ID NO: 1 with a leucine | 1574 |
| Mutation predicted to result in substitution of | Yarbrough et al., Journal of the |
| histidine corresponding to amino acid position 83 of | National Cancer Institute, 91(18): 1569- |
| SEQ ID NO: 1 with a tyrosine | 1574 |

The disclosure also features a method of treating a human subject having, suspected of having, or at risk of developing a disease or disorder associated with CDK2, comprising: (i) identifying, in a biological sample obtained from the human subject: (a) a nucleotide sequence encoding a p16 protein comprising the amino acid sequence of SEQ ID NO:1, (b) a CDKN2A gene lacking one or more inactivating nucleic acid substitutions, and/or (c) the presence of a p16 protein; (ii) identifying, in a biological sample obtained from the human subject: (a) an amplification of the CCNE1 gene and/or (b) an expression level of CCNE1 that is higher than a control expression level of CCNE1; and (iii) administering a CDK2 inhibitor to the human subject. In some embodiments, the subject has a disease or disorder associated with CDK2. In some embodiments, the subject is suspected of having or is at risk of developing a disease or disorder associated with CDK2. In some embodiments, the method comprises: (i) identifying, in a biological sample obtained from the human subject: (a) a nucleotide sequence encoding a p16 protein comprising the amino acid sequence of SEQ ID NO:1, (b) a CDKN2A gene lacking one or more inactivating nucleic acid substitutions and/or deletions, and/or (c) the presence of a p16 protein; (ii) identifying, in a biological sample obtained from the human subject: (a) an amplification of the CCNE1 gene; and (iii) administering a CDK2 inhibitor to the human subject.

The disclosure also features a method of predicting the response of a human subject having, suspected of having, or at risk of developing a disease or disorder associated with CDK2 to a CDK2 inhibitor, comprising: (i) determining, from a biological sample obtained from the human subject: (a) the nucleotide sequence of a CDKN2A gene, (b) the presence of a CDKN2A gene lacking one or more inactivating nucleic acid substitutions and/or deletions, and/or (c) the presence of a p16 protein; and (ii) determining, from a biological sample obtained from the human subject: (a) the copy number of the CCNE1 gene and/or (b) the expression level of CCNE1, wherein (1) (a) the presence of a CDKN2A gene encoding a p16 protein comprising the amino acid sequence of SEQ ID NO:1, (b) the presence of a CDKN2A gene lacking one or more inactivating nucleic acid substitutions and/or deletions, and/or (c) the presence of a p16 protein, and (2) (a) an amplification of the CCNE1 gene and/or (b) an expression level of CCNE1 that is higher than a control expression level of CCNE1, is predictive that the human subject will respond to the CDK2 inhibitor. In some embodiments, the subject has a disease or disorder associated with CDK2. In some embodiments, the subject is suspected of having or is at risk of developing a disease or disorder associated with CDK2. In some embodiments, the method comprises: (i) determining, from a biological sample obtained from the human subject: (a) the nucleotide sequence of a CDKN2A gene and/or (b) the presence of a CDKN2A gene lacking one or more inactivating nucleic acid substitutions and/or deletions; and (ii) determining, from a biological sample obtained from the human subject: (a) the copy number of the CCNE1 gene, wherein (1) (a) the presence of a CDKN2A gene encoding a p16 protein comprising the amino acid sequence of SEQ ID NO:1 and/or (b) the presence of a CDKN2A gene lacking one or more inactivating nucleic acid substitutions and/or deletions, and (2) (a) an amplification of the CCNE1 gene, is predictive that the human subject will respond to the CDK2 inhibitor.

In specific embodiments, the (i) determining of (a) the nucleotide sequence of a CDKN2A gene, (b) the presence of a CDKN2A gene lacking one or more inactivating nucleic acid substitutions and/or deletions, and/or (c) the presence of a p16 protein is performed before (e.g., at least 1 day, at least 2 days, at least 3 days, at least 4 days, at least 5 days, at least 6 days, at least 7 days, at least 2 weeks, at least 3 weeks, or at least 4 weeks, or from 6 hours to 16 hours, from 6 hours to 20 hours, or from 6 hours to 24 hours, from 2 days to 3 days, from 2 days to 4 days, from 2 days to 5 days, from 2 days to 6 days, from 2 days to 7 days, from 1 week to 2 weeks, from 1 week to 3 weeks, or from 1 week to 4 weeks before) administering to the human subject the CDK2 inhibitor. In specific embodiments, the (ii) determining of (a) the copy number of the CCNE1 gene and/or (b) the expression level of CCNE1 in the biological sample obtained from the human subject is performed before (e.g., at least 1 day, at least 2 days, at least 3 days, at least 4 days, at least 5 days, at least 6 days, at least 7 days, at least 2 weeks, at least 3 weeks, or at least 4 weeks, or from 6 hours to 16 hours, from 6 hours to 20 hours, or from 6 hours to 24 hours, from 2 days to 3 days, from 2 days to 4 days, from 2 days to 5 days, from 2 days to 6 days, from 2 days to 7 days, from 1 week to 2 weeks, from 1 week to 3 weeks, or from 1 week to 4 weeks before) administering to the human subject the CDK2 inhibitor.

An amplification of the CCNE1 gene and/or an expression level of CCNE1 that is higher than a control expression level of CCNE1, combined with the presence of a CDKN2A gene encoding a p16 protein comprising the amino acid sequence of SEQ ID NO:1, the presence of a CDKN2A gene lacking one or more inactivating nucleic acid substitutions and/or deletions, and/or the presence of a p16 protein (e.g., a p16 protein comprising the amino acid sequence of SEQ ID NO:1), is indicative/predictive that a human subject having, suspected of having, or at risk of developing a disease or disorder associated with CDK2 will respond to a CDK2 inhibitor.

In some embodiments, the CCNE1 gene is amplified to a gene copy number from 3 to 25. In specific embodiments, the CCNE1 gene is amplified to a gene copy number of at least 3. In specific embodiments, the CCNE1 gene is amplified to a gene copy number of at least 5. In specific embodiments, the CCNE1 gene is amplified to a gene copy number of at least 7. In specific embodiments, the CCNE1 gene is amplified to a gene copy number of at least 10. In specific embodiments, the CCNE1 gene is amplified to a gene copy number of at least 12. In specific embodiments, the CCNE1 gene is amplified to a gene copy number of at least 14. In specific embodiments, the CCNE1 gene is amplified to a gene copy number of at least 21.

In specific embodiments, the expression level of CCNE1 is the level of CCNE1 mRNA. In specific embodiments, the expression level of CCNE1 is the level of CCNE1 protein.

In some embodiments of the foregoing methods, the control expression level of CCNE1 is a pre-established cut-off value. In some embodiments of the foregoing methods, the control expression level of CCNE1 is the expression level of CCNE1 in a sample or samples obtained from one or more subjects that have not responded to treatment with the CDK2 inhibitor.

In some embodiments of the foregoing methods, the expression level of CCNE1 is the expression level of CCNE1 mRNA. In some embodiments of the foregoing methods, the expression level of CCNE1 is the expression level of CCNE1 protein. In some embodiments in which the expression level of CCNE1 is the expression level of CCNE1 mRNA, the expression level of CCNE1 is measured by RNA sequencing, quantitative polymerase chain reaction (PCR), in situ hybridization, nucleic acid array or RNA sequencing. In some embodiments in which the expression level of CCNE1 is the expression level of CCNE1 protein, the expression level of CCNE1 is measured by western blot, enzyme-linked immunosorbent assay, or immunohistochemistry staining.

### Rb S780

The disclosure also features a method for assessing the CDKN2A gene and the CCNE1 gene, comprising determining, from a biological sample or biological samples obtained from a human subject having a disease or disorder associated with CDK2, (i) (a) the nucleotide sequence of a CDKN2A gene or (b) the presence of a CDKN2A gene lacking one or more inactivating nucleic acid substitutions and/or deletions, and (ii) the copy number of the CCNE1 gene.

The disclosure also features a method of evaluating the response of a human subject having, suspected of having, or at risk of developing a disease or disorder associated with CDK2 to a CDK2 inhibitor, comprising: (a) administering a CDK2 inhibitor to the human subject, wherein the human subject has been previously determined to have an amplification of the CCNE1 gene and/or an expression level of CCNE1 that is higher than a control expression level of CCNE1; (b) measuring, in a biological sample of obtained from the subject subsequent to the administering of step (a), the level of retinoblastoma (Rb) protein phosphorylation at the serine corresponding to amino acid position 780 of SEQ ID NO:3, wherein a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, is indicative that the human subject responds to the CDK2 inhibitor. In some embodiments, the subject has a disease or disorder associated with CDK2. In some embodiments, the subject is suspected of having or is at risk of developing a disease or disorder associated with CDK2. In some embodiments, the biological sample comprises a blood sample or a tumor biopsy sample.

Phosphorylation of Rb at the serine corresponding to amino acid position 780 of SEQ ID NO:3 (referred to herein as “Ser780” or “S780”) has been identified in the Examples as a pharmacodynamic marker useful in assessing responsiveness (e.g., inhibition by CDK2) of a human subject having a disease or disorder having CCNE1 amplification to a CDK2 inhibitor.

Rb is a regulator of the cell cycle and acts as a tumor suppressor. Rb is activated upon phosphorylation by cyclin D-CDK4/6 at Ser780 and Ser795 and by cyclin E/CDK2 at Ser807 and Ser811. Rb is encoded by the RB transcriptional corepressor 1 (“RB1”) gene (GenBank Accession No. NM_000321). The amino acid sequence of human Rb is provided below (GenBank Accession No. NP_000312/UniProtKB Accession No. P06400) (5780 is in bold and underlined):

| (SEQ ID NO: 3) | |
|---|---|
| 1 | MPPKTPRKTA ATAAAAAAEP PAPPPPPPPE EDPEQDSGPE DLPLVRLEFE ETEEPDFTAL |
| 61 | CQKLKIPDHV RERAWLTWEK VSSVDGVLGG YIQKKKELWG ICIFIAAVDL DEMSFTFTEL |
| 121 | QKNIEISVHK FFNLLKEIDT STKVDNAMSR LLKKYDVLFA LFSKLERTCE LIYLTQPSSS |
| 181 | ISTEINSALV LKVSWITFLL AKGEVLQMED DLVISFQLML CVLDYFIKLS PPMLLKEPYK |
| 241 | TAVIPINGSP RTPRRGQNRS ARIAKQLEND TRIIEVLCKE HECNIDEVKN VYFKNFIPFM |
| 301 | NSLGLVTSNG LPEVENLSKR YEEIYLKNKD LDARLFLDHD KTLQTDSIDS FETQRTPRKS |
| 361 | NLDEEVNVIP PHTPVRTVMN TIQQLMMILN SASDQPSENL ISYFNNCTVN PKESILKRVK |
| 421 | DIGYIFKEKF AKAVGQGCVE IGSQRYKLGV RLYYRVMESM LKSEEERLSI QNFSKLLNDN |
| 481 | IFHMSLLACA LEVVMATYSR STSQNLDSGT DLSFPWILNV LNLKAFDFYK VIESFIKAEG |
| 541 | NLTREMIKHL ERCEHRIMES LAWLSDSPLF DLIKQSKDRE GPTDHLESAC PLNLPLQNNH |
| 601 | TAADMYLSPV RSPKKKGSTT RVNSTANAET QATSAFQTQK PLKSTSLSLF YKKVYRLAYL |
| 661 | RLNTLCERLL SEHPELEHII WTLFQHTLQN EYELMRDRHL DQIMMCSMYG ICKVKNIDLK |
| 721 | FKIIVTAYKD LPHAVQETFK RVLIKEEEYD SIIVFYNSVF MQRLKTNILQ YASTRPPTLS |
| 781 | PIPHIPRSPY KFPSSPLRIP GGNIYISPLK SPYKISEGLP TPTKMTPRSR ILVSIGESFG |
| 841 | TSEKFQKINQ MVCNSDRVLK RSAEGSNPPK PLKKLRFDIE GSDEADGSKH LPGESKFQQK |
| 901 | LAEMTSTRTR MQKQKMNDSM DTSNKEEK. |

As stated above, the Examples demonstrate CDK2-knockdown inhibits proliferation in CCNE1-amplified cell lines, but not in CCNE1-non-amplified cell lines. The Examples further demonstrate CDK2-knockdown or inhibition blocks Rb phosphorylation at the S780 in CCNE1-amplified cell lines, but not in CCNE1-non-amplified cell lines. Accordingly, Rb phosphorylation at the serine corresponding to amino acid position 780 of SEQ ID NO:3 is a pharmacodynamic marker for assessing response to CDK2 inhibition in CCNE1 amplified cancer cells or patients with diseases or disorders having CCNE1 amplification. Thus, provided herein are methods relating to the use of the level of Rb phosphorylation at the serine corresponding to amino acid position 780 of SEQ ID NO:3 in a human subject having, suspected of having, or at risk of developing a disease or disorder associated with CDK2 as a marker for indicating the response of the human subject to a CDK2 inhibitor, wherein the human subject has an increased expression level of CCNE1.

Thus, the disclosure features a method for measuring the amount of a protein in a sample, comprising: (a) providing a biological sample obtained from a human subject having a disease or disorder associated with CDK2; and (b) measuring the level of Rb protein phosphorylation at the serine corresponding to amino acid position 780 of SEQ ID NO:3 in the biological sample. In some embodiments, the biological sample comprises a blood sample or a tumor biopsy sample. In a specific embodiment, provided herein is a method of evaluating the response of a human subject having, suspected of having, or at risk of developing a disease or disorder associated with CDK2 to a CDK2 inhibitor, comprising: (a) administering a CDK2 inhibitor to the human subject, wherein the human subject has been previously determined to have an amplification of the CCNE1 gene and/or an expression level of CCNE1 that is higher than a control expression level of CCNE1; and (b) measuring, in a biological sample obtained from the human subject subsequent to the administering of step (a), the level of Rb phosphorylation at the serine corresponding to amino acid position 780 of SEQ ID NO:3, wherein a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, is indicative that the human subject responds to the CDK2 inhibitor. In specific embodiments, the human subject has a disease or disorder associated with CDK2.

A reduced level of Rb phosphorylation at the serine corresponding to amino acid position 780 of SEQ ID NO:3, as compared to a control level of Rb phosphorylation at the serine corresponding to amino acid position 780 of SEQ ID NO:3, combined with an amplification of the CCNE1 gene and/or an expression level of CCNE1 that is higher than a control expression level of CCNE1, is indicative that a human subject having, suspected of having, or at risk of developing a disease or disorder associated with CDK2 responds to a CDK2 inhibitor. For example, in a subject having an amplification of the CCNE1 gene and/or an expression level of CCNE1 that is higher than a control expression level of CCNE1, a biological sample, obtained from the subject after treatment with a CDK2 inhibitor, having low (e.g., reduced as compared to a control) or undetectable levels of Rb phosphorylation at serine corresponding to amino acid position 780 of SEQ ID NO:3 is indicative that the subject responds to the CDK2 inhibitor.

A biological sample, obtained from a subject after administration of a CDK2 inhibitor to the subject, having a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, combined with: (i) an amplification of the CCNE1 gene and/or an expression level of CCNE1 that is higher than a control expression level of CCNE1, and (ii) presence of a CDKN2A gene encoding a p16 protein comprising the amino acid sequence of SEQ ID NO:1, presence of a CDKN2A gene lacking one or more inactivating nucleic acid substitutions and/or deletions, and/or presence of a p16 protein (e.g., a p16 protein comprising the amino acid sequence of SEQ ID NO:1), is indicative that a human subject having, suspected of having, or at risk of developing a disease or disorder associated with CDK2 responds to a CDK2 inhibitor. For example, in a human subject having (i) an amplification of the CCNE1 gene and/or an expression level of CCNE1 that is higher than a control expression level of CCNE1, and (ii) the presence of a CDKN2A gene encoding a p16 protein comprising the amino acid sequence of SEQ ID NO:1, the presence of a CDKN2A gene lacking one or more inactivating nucleic acid substitutions and/or deletions, and/or the presence of a p16 protein (e.g., a p16 protein comprising the amino acid sequence of SEQ ID NO:1), a biological sample, obtained from the human subject after administration of a CDK2 inhibitor to the subject, having low (e.g., reduced as compared to a control) or undetectable levels of Rb phosphorylation at the serine corresponding to amino acid position 780 of SEQ ID NO:3 is indicative that the human subject responds to the CDK2 inhibitor.

In some embodiments, the CCNE1 gene is amplified to a gene copy number from 3 to 25. In specific embodiments, the CCNE1 gene is amplified to a gene copy number of at least 3. In specific embodiments, the CCNE1 gene is amplified to a gene copy number of at least 5. In specific embodiments, the CCNE1 gene is amplified to a gene copy number of at least 7. In specific embodiments, the CCNE1 gene is amplified to a gene copy number of at least 10. In specific embodiments, the CCNE1 gene is amplified to a gene copy number of at least 12. In specific embodiments, the CCNE1 gene is amplified to a gene copy number of at least 14. In specific embodiments, the CCNE1 gene is amplified to a gene copy number of at least 21. In specific embodiments, the expression level of CCNE1 is the level of CCNE1 mRNA. In specific embodiments, the expression level of CCNE1 is the level of CCNE1 protein.

### Controls

As described above, the methods related to biomarkers and pharmacodynamic markers can involve, measuring one or more markers (e.g., a biomarker or a pharmacodynamics marker, e.g., the amplification of the CCNE1 gene, the expression level of CCNE1, the presence of a CDKN2A gene encoding a p16 protein comprising the amino acid sequence of SEQ ID NO:1, the presence of a CDKN2A gene lacking one or more inactivating nucleic acid substitutions and/or deletions, the presence of a p16 protein (e.g., a p16 protein comprising the amino acid sequence of SEQ ID NO:1), and Rb phosphorylation at the serine corresponding to amino acid position 780 of SEQ ID NO:3) in a biological sample from a human subject having, suspected of having or at risk of developing a disease or disorder associated with CDK2. In specific embodiments, the human subject has a disease or disorder associated with CDK2. In specific embodiments, the human subject is suspected of having or is at risk of developing a disease or disorder associated with CDK2. In certain aspects, the level (e.g., amplification (e.g., for the CCNE1 gene), expression level (e.g., for CCNE1 or p16 protein), or phosphorylation level (e.g., for Rb)) of one or more biomarkers, compared to a control level of the one or more biomarkers, predicts/indicates the response of a human subject to treatment comprising a CDK2 inhibitor. In certain embodiments, when (i) the CCNE1 gene is amplified and/or an expression level of CCNE1 that is higher than a control expression level of CCNE1, and (ii) a CDKN2A gene encoding a p16 protein comprising the amino acid sequence of SEQ ID NO:1 is present, a CDKN2A gene lacking one or more inactivating nucleic acid substitutions and/or deletions is present, and/or a p16 protein (e.g., a p16 protein comprising the amino acid sequence of SEQ ID NO:1) is present, the human subject is identified as likely to respond to a CDK2 inhibitor. In other embodiments, when (i) the CCNE1 gene is amplified and/or an expression level of CCNE1 that is higher than a control expression level of CCNE1, and (ii) in a biological sample from the human subject after the human subject has been administered a CDK2 inhibitor, the level of Rb phosphorylation at the serine corresponding to amino acid position 780 of SEQ ID NO:3 is less than the control level of Rb phosphorylation at the serine corresponding to amino acid position 780 of SEQ ID NO:3, the human subject is identified as responding to a CDK2 inhibitor. In yet another embodiment, when (i) the CCNE1 gene is amplified and/or an expression level of CCNE1 that is higher than a control expression level of CCNE1, (ii) a CDKN2A gene encoding a p16 protein comprising the amino acid sequence of SEQ ID NO:1 is present, a CDKN2A gene lacking one or more inactivating nucleic acid substitutions and/or deletions is present, and/or a p16 protein (e.g., a p16 protein comprising the amino acid sequence of SEQ ID NO:1) is present, and (iii) in a biological sample from the human subject after the human subject has been administered a CDK2 inhibitor, the level of Rb phosphorylation at the serine corresponding to amino acid position 780 of SEQ ID NO:3 is less than the control level of Rb phosphorylation at the serine corresponding to amino acid position 780 of SEQ ID NO:3, the human subject is identified as responding to a CDK2 inhibitor. In this context, the term “control” includes a sample (from the same tissue type) obtained from a human subject who is known to not respond to a CDK2 inhibitor. The term “control” also includes a sample (from the same tissue type) obtained in the past from a human subject who is known to not respond to a CDK2 inhibitor and used as a reference for future comparisons to test samples taken from human subjects for which therapeutic responsiveness is to be predicted. The “control” level (e.g., gene copy number, expression level, or phosphorylation level) for a particular biomarker (e.g., CCNE1, p16, or Rb phosphorylation) in a particular cell type or tissue may be pre-established by an analysis of biomarker level (e.g., expression level or phosphorylation level) in one or more (e.g., 2, 3, 4, 5, 6, 7, 8, 9, 10, 15, 20, 25, 30, 35, or 40 or more) human subjects that have not responded to treatment with a CDK2 inhibitor. This pre-established reference value (which may be an average or median level (e.g., gene copy number, expression level, or phosphorylation level) taken from multiple human subjects that have not responded to the therapy) may then be used for the “control” level of the biomarker (e.g., CCNE1, p16, or Rb phosphorylation) in the comparison with the test sample. In such a comparison, the human subject is predicted to respond to a CDK2 inhibitor if the CCNE1 gene is amplified and/or the expression level of CCNE is higher than the pre-established reference, and a CDKN2A gene encoding a p16 protein comprising the amino acid sequence of SEQ ID NO:1 is present, a CDKN2A gene lacking one or more inactivating nucleic acid substitutions and/or deletions is present, and/or a p16 protein (e.g., a p16 protein comprising the amino acid sequence of SEQ ID NO:1) is present. In another such a comparison, the human subject is predicted to respond to a CDK2 inhibitor if (i) CCNE1 gene is amplified and/or the expression level of CCNE is higher than the pre-established reference, and (ii) after administering to the human subject a CDK2 inhibitor, the level of Rb phosphorylation at the serine corresponding to amino acid position 780 of SEQ ID NO:3 is lower than the pre-established reference. In yet another such a comparison, the human subject is indicated to respond to a CDK2 inhibitor if (i) CCNE1 gene is amplified and/or the expression level of CCNE is higher than the pre-established reference, (ii) a CDKN2A gene encoding a p16 protein comprising the amino acid sequence of SEQ ID NO:1 is present, a CDKN2A gene lacking one or more inactivating nucleic acid substitutions and/or deletions is present, and/or a p16 protein (e.g., a p16 protein comprising the amino acid sequence of SEQ ID NO:1) is present, and (iii) after administering to the human subject a CDK2 inhibitor, the level of Rb phosphorylation at the serine corresponding to amino acid position 780 of SEQ ID NO:3 is lower than the pre-established reference.

The “control” level for a particular biomarker in a particular cell type or tissue may alternatively be pre-established by an analysis of biomarker level in one or more human subjects that have responded to treatment with a CDK2 inhibitor. This pre-established reference value (which may be an average or median level (e.g., expression level or phosphorylation level) taken from multiple human subjects that have responded to the therapy) may then be used as the “control” level (e.g., expression level or phosphorylation level) in the comparison with the test sample. In such a comparison, the human subject is indicated to respond to a CDK2 inhibitor if the level (e.g., copy number of the CCNE1 gene, expression level of CCNE1, expression level of p16, or phosphorylation level of Rb at the serine corresponding to amino acid position 780 of SEQ ID NO:3) of the biomarker being analyzed is equal or comparable to (e.g., at least 85% but less than 115% of), the pre-established reference.

In certain embodiments, the “control” is a pre-established cut-off value. A cut-off value is typically a level (e.g., a copy number, an expression level, or a phosphorylation level) of a biomarker above or below which is considered predictive of responsiveness of a human subject to a therapy of interest. Thus, in accordance with the methods and compositions described herein, a reference level (e.g., of CCNE1 gene copy number, CCNE1 expression, p16 expression, or Rb phosphorylation at the serine corresponding to amino acid position 780 of SEQ ID NO:3) is identified as a cut-off value, above or below of which is predictive of responsiveness to a CDK2 inhibitor. Cut-off values determined for use in the methods described herein can be compared with, e.g., published ranges of concentrations but can be individualized to the methodology used and patient population.

In some embodiments, the expression level of CCNE1 is increased as compared to the expression level of CCNE1 in a control. For example, the expression level of CCNE1 analyzed can be at least 1.5, at least 2, at least 3, at least 4, at least 5, at least 6, at least 7, at least 8, at least 9, at least 10, at least 20, at least 25, at least 50, at least 75, or at least 100 times higher, or at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1,000%, at least 1,500%, at least 2,000%, at least 2,500%, at least 3,000%, at least 3,500%, at least 4,000%, at least 4,500%, or at least 5,000% higher, than the expression level of CCNE1 in a control.

A p16 protein is present if the protein is detectable by any assay known in the art or described herein, such as, for example, western blot, immunohistochemistry, fluorescence-activated cell sorting, and enzyme-linked immunoassay. In some embodiments, a p16 protein is present at an expression level that is within at least 5%, at least 10%, at least 20%, or at least 30% of the p16 expression level in a healthy control.

In some embodiments, the level of Rb phosphorylation at the serine corresponding to amino acid position 780 of SEQ ID NO:3 being analyzed is reduced as compared to the level of Rb phosphorylation at the serine corresponding to amino acid position 780 of SEQ ID NO:3 in a control. For example, the level of the Rb phosphorylation at the serine corresponding to amino acid position 780 of SEQ ID NO:3 being analyzed can be at least 1.5, at least 2, at least 3, at least 4, at least 5, at least 6, at least 7, at least 8, at least 9, at least 10, at least 20, at least 25, at least 50, at least 75, or at least 100 times lower, or at least 10%, at least 20%, at least 30%, at least 40%, at least 50%, at least 60%, at least 70%, at least 80%, at least 90%, or 100% lower, than the level of Rb phosphorylation at the serine corresponding to amino acid position 780 of SEQ ID NO:3 in a control.

### Biological Samples

Suitable biological samples for the methods described herein include any sample that contains blood or tumor cells obtained or derived from the human subject in need of treatment. For example, a biological sample can contain tumor cells from biopsy from a patient suffering from a solid tumor. A tumor biopsy can be obtained by a variety of means known in the art. Alternatively, a blood sample can be obtained from a patients suffering from a hematological cancer.

A biological sample can be obtained from a human subject having, suspected of having, or at risk of developing, a disease or disorder associated with CDK2. In some embodiments, the disease or disorder associated with CDK2 is a cancer (such as those described supra).

Methods for obtaining and/or storing samples that preserve the activity or integrity of molecules (e.g., nucleic acids or proteins) in the sample are well known to those skilled in the art. For example, a biological sample can be further contacted with one or more additional agents such as buffers and/or inhibitors, including one or more of nuclease, protease, and phosphatase inhibitors, which preserve or minimize changes in the molecules in the sample.

### Evaluating Biomarkers and Pharmacodynamic Markers

Expression levels of CCNE1 or p16 can be detected as, e.g., RNA expression of a target gene (i.e., the genes encoding CCNE1 or p16). That is, the expression level (amount) of CCNE1 or p16 can be determined by detecting and/or measuring the level of mRNA expression of the gene encoding CCNE1. Alternatively, expression levels of CCNE1 or p16 can be detected as, e.g., protein expression of target gene (i.e., the genes encoding CCNE1 or p16). That is, the expression level (amount) of CCNE1 or p16 can be determined by detecting and/or measuring the level of protein expression of the genes encoding CCNE1 or p16.

In some embodiments, the expression level of CCNE1 or p16 is determined by measuring RNA levels. A variety of suitable methods can be employed to detect and/or measure the level of mRNA expression of a gene. For example, mRNA expression can be determined using Northern blot or dot blot analysis, reverse transcriptase-PCR (RT-PCR; e.g., quantitative RT-PCR), in situ hybridization (e.g., quantitative in situ hybridization), nucleic acid array (e.g., oligonucleotide arrays or gene chips) and RNA sequencing analysis. Details of such methods are described below and in, e.g., Sambrook et al., Molecular Cloning: A Laboratory Manual Second Edition vol. 1, 2 and 3. Cold Spring Harbor Laboratory Press: Cold Spring Harbor, New York, USA, Nov. 1989; Gibson et al. (1999) Genome Res., 6(10):995-1001; and Zhang et al. (2005) Environ. Sci. Technol., 39(8):2777-2785; U.S. Publication No. 2004086915; European Patent No. 0543942; and U.S. Pat. No. 7,101,663; Kukurba et al. (2015) Cold Spring Harbor Protocols., 2015 (11): 951-69; the disclosures of each of which are incorporated herein by reference in their entirety.

In one example, the presence or amount of one or more discrete mRNA populations in a biological sample can be determined by isolating total mRNA from the biological sample (see, e.g., Sambrook et al. (supra) and U.S. Pat. No. 6,812,341) and subjecting the isolated mRNA to agarose gel electrophoresis to separate the mRNA by size. The size-separated mRNAs are then transferred (e.g., by diffusion) to a solid support such as a nitrocellulose membrane. The presence or amount of one or more mRNA populations in the biological sample can then be determined using one or more detectably-labeled-polynucleotide probes, complementary to the mRNA sequence of interest, which bind to and thus render detectable their corresponding mRNA populations. Detectable-labels include, e.g., fluorescent (e.g., umbelliferone, fluorescein, fluorescein isothiocyanate, rhodamine, dichlorotriazinylamine fluorescein, dansyl chloride, allophycocyanin, or phycoerythrin), luminescent (e.g., europium, terbium, Qdot™ nanoparticles supplied by the Quantum Dot Corporation, Palo Alto, CA), radiological (e.g., ¹²⁵I, ¹³¹I, ³⁵S, ³²P, ³³P or ³H), and enzymatic (horseradish peroxidase, alkaline phosphatase, beta-galactosidase, or acetylcholinesterase) labels.

In some embodiments, the expression level of CCNE1 or p16 is determined by measuring protein levels. A variety of suitable methods can be employed to detect and/or measure the level of protein expression of target genes. For example, CCNE1 or p16 protein expression can be determined using western blot, enzyme-linked immunosorbent assay (“ELISA”), fluorescence activated cell sorting, or immunohistochemistry analysis (e.g., using a CCNE1-specific or p16-specific antibody, respectively). Details of such methods are described below and in, e.g., Sambrook et al., supra.

In one example, the presence or amount of one or more discrete protein populations (e.g., CCNE1 or p16) in a biological sample can be determined by western blot analysis, e.g., by isolating total protein from the biological sample (see, e.g., Sambrook et al. (supra)) and subjecting the isolated protein to agarose gel electrophoresis to separate the protein by size. The size-separated proteins are then transferred (e.g., by diffusion) to a solid support such as a nitrocellulose membrane. The presence or amount of one or more protein populations in the biological sample can then be determined using one or more antibody probes, e.g., a first antibody specific for the protein of interest (e.g., CCNE1 or p16), and a second antibody, detectably labeled, specific for the first antibody, which binds to and thus renders detectable the corresponding protein population. Detectable-labels suitable for use in western blot analysis are known in the art.

Methods for detecting or measuring gene expression (e.g., mRNA or protein expression) can optionally be performed in formats that allow for rapid preparation, processing, and analysis of multiple samples. This can be, for example, in multi-welled assay plates (e.g., 96 wells or 386 wells) or arrays (e.g., nucleic acid chips or protein chips). Stock solutions for various reagents can be provided manually or robotically, and subsequent sample preparation (e.g., RT-PCR, labeling, or cell fixation), pipetting, diluting, mixing, distribution, washing, incubating (e.g., hybridization), sample readout, data collection (optical data) and/or analysis (computer aided image analysis) can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay. Exemplary high-throughput cell-based assays (e.g., detecting the presence or level of a target protein in a cell) can utilize ArrayScan® VTI HCS Reader or KineticScan® HCS Reader technology (Cellomics Inc., Pittsburgh, PA).

In some embodiments, the presence of a CDKN2A gene encoding a p16 protein comprising the amino acid sequence of SEQ ID NO:1 and/or the presence of a CDKN2A gene lacking one or more inactivating nucleic acid substitutions and/or deletions is determined by evaluating the DNA sequence of the CDKN2A gene (e.g., genomic DNA or cDNA) or by evaluating the RNA sequence of the CDKN2A gene (e.g., RNA, e.g., mRNA). Methods of performing nucleic acid sequencing analyses are known in the art and described above. Nonlimiting examples of inactivating nucleic acid substitutions and/or deletions preventing the CDKN2A gene from encoding a protein comprising the amino acid sequence of SEQ ID NO:1 are described in Table A, above. In specific embodiments, the one or more inactivating nucleic acid substitutions and/or deletions in the CDKN2A gene is as described in Yarbrough et al., Journal of the National Cancer Institute, 91(18):1569-1574, 1999; Liggett and Sidransky, Biology of Neoplasia, Journal of Oncology, 16(3):1197-1206, 1998, and Cairns et al., Nature Genetics, 11:210-212, 1995, each of which is incorporated by reference herein in its entirety.

In some embodiments, the expression level of a gene or the presence of a gene lacking one or more inactivating nucleic acid substitutions or deletions is determined by evaluating the copy number variation (CNV) of the gene. The CNV of genes (e.g., the CCNE1 gene and/or the CDKN2A gene) can be determined/identified by a variety of suitable methods. For example, CNV can be determined using fluorescent in situ hybridization (FISH), multiplex ligation dependent probe amplification (MLPA), array comparative genomic hybridization (aCGH), single-nucleotide polymorphisms (SNP) array, and next-generation sequencing (NGS) technologies.

In one example, the copy number variation of one or more discrete genes in a biological sample can be determined by MLPA, e.g., by extracting DNA specimens from the biological sample (see, e.g., Sambrook et al. (supra) and U.S. Pat. No. 6,812,341), and amplifying DNA sequence of interest (e.g., CCNE1 or CDKN2A) using a mixture of MLPA probes. Each MLPA probe consists of two oligonucleotides that hybridize to immediately adjacent target DNA sequence (e.g., CCNE1 or CDKN2A) in order to be ligated into a single probe. Ligated probes are amplified though PCR with one PCR primer fluorescently labeled, enabling the amplification products to be visualized during fragment separation by capillary electrophoresis. The presence, absence or amplification of one or more genes of interest in the biological sample is calculated by measuring PCR derived fluorescence, quantifying the amount of PCR product after normalization and comparing it with control DNA samples.

The level of Rb phosphorylation at the serine corresponding to amino acid position 780 of SEQ ID NO:3 can be detected by a variety of suitable methods. For example, phosphorylation status can be determined using western blot, ELISA, fluorescence activated cell sorting, or immunohistochemistry analysis. Details of such methods are described below and in, e.g., Sambrook et al., supra.

As with the methods for detecting or measuring gene expression (above), methods for detecting or measuring the level of Rb phosphorylation at the serine corresponding to amino acid position 780 of SEQ ID NO:3 can optionally be performed in formats that allow for rapid preparation, processing, and analysis of multiple samples.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g., “Two-Pump at-Column Dilution Configuration for Preparative LC-MS,” K. Blom, J. Combi. Chem., 4, 295 (2002); “Optimizing Preparative LC-MS Configurations and Methods for Parallel Synthesis Purification,” K. Blom, R. Sparks, J. Doughty, G. Everlof, T. Haque, A. Combs, J. Combi. Chem., 5, 670 (2003); and “Preparative LC-MS Purification: Improved Compound Specific Method Optimization,” K. Blom, B. Glass, R. Sparks, A. Combs, J. Combi. Chem., 6, 874-883 (2004). The separated compounds were typically subjected to analytical liquid chromatography mass spectrometry (LCMS) for purity check under the following conditions: Instrument: Agilent 1100 series, LC/MSD; Column: Waters Sunfire™ C₁₈ 5 μm particle size, 2.1×5.0 mm; Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:
pH=2 purifications: Waters Sunfire™ C₁₈ 5 μm particle size, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature (see “Preparative LCMS Purification: Improved Compound Specific Method Optimization,” K. Blom, B. Glass, R. Sparks, A. Combs, J. Comb. Chem., 6, 874-883 (2004)). Typically, the flow rate used with the 30×100 mm column was 60 mL/minute.
pH=10 purifications: Waters XBridge C₁₈ 5 μm particle size, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature (See “Preparative LCMS Purification: Improved Compound Specific Method Optimization,” K. Blom, B. Glass, R. Sparks, A. Combs, J. Comb. Chem., 6, 874-883 (2004)). Typically, the flow rate used with 30×100 mm column was 60 mL/minute.

### Intermediate 1. 7-(1-(1-Ethoxyethyl)-1H-pyrazol-4-yl)-8-isopropoxy-[1,2,4]triazolo[1,5-c]pyrimidin-2-amine

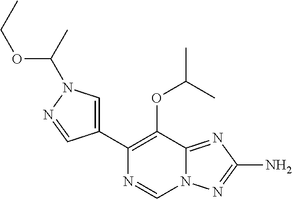 [SMILES: CCOC(C)n1cc(-c2ncn3nc(N)nc3c2OC(C)C)cn1]

### Step 1: 4-Amino-6-chloropyrimidin-5-ol hydrobromide

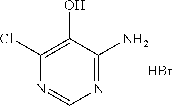

To a mixture of 6-chloro-5-methoxypyrimidin-4-amine (26.0 g, 163 mmol, Combi-Blocks QC-2900) in CH₂Cl₂ (500 mL) was slowly added neat boron tribromide (17.7 mL, 187 mmol) and the reaction mixture was stirred at r.t. for 3 days. The reaction mixture was quenched with 2-propanol (43.9 mL, 570 mmol) and Et₂O (200 mL) was added. The solid precipitate that formed was collected via filtration, washed with Et₂O, and dried under vacuum to afford the desired product as the HBr salt. The crude material obtained was used directly without further purification. LC-MS calculated for C₄H₅ClN₃O (M+H)⁺: m/z=146.0; found 145.9.

### Step 2: 6-Chloro-5-isopropoxypyrimidin-4-amine

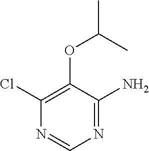

To a mixture of 4-amino-6-chloropyrimidin-5-ol hydrobromide (Step 1) in CH₃CN (300 mL) was added 2-iodopropane (25.0 mL, 250 mmol) and K₂CO₃ (56.3 g, 407 mmol) and the reaction mixture was stirred at 60° C. overnight. After cooling to r.t., the reaction mixture was diluted with EtOAc (300 mL) and filtered. The filtrate was concentrated under reduced pressure, and the crude residue was dissolved in EtOAc and the mixture was concentrated to half volume before hexanes was added. The solid precipitate that formed was collected via filtration, washed with 10% EtOAc in hexanes, and dried under vacuum to afford the desired product. The crude material obtained was used directly without further purification. LC-MS calculated for C₇H₁₁ClN₃O (M+H)⁺: m/z=188.1; found 188.0.

### Step 3: 6-(1-(1-Ethoxyethyl)-1H-pyrazol-4-yl)-5-isopropoxypyrimidin-4-amine

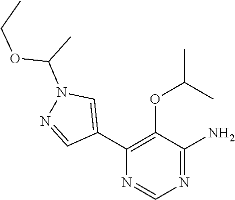 [SMILES: CCOC(C)n1cc(-c2ncnc(N)c2OC(C)C)cn1]

A mixture of 6-chloro-5-isopropoxypyrimidin-4-amine (7.45 g, 39.7 mmol), 1-(1-ethoxyethyl)-4-(4,4,5,5-tetramethyl-1,3,2-dioxaborolan-2-yl)-1H-pyrazole (12.7 g, 48 mmol, Combi-Blocks FM-3403), Pd(dppf)Cl₂ CH₂Cl₂ adduct (1.62 g, 1.98 mmol), and K₃PO₄ (25.3 g, 119 mmol) in 1,4-dioxane/H₂O (5:1, 200 mL) was purged with N₂ and stirred at 90° C. overnight. After cooling to r.t., the reaction mixture was diluted with water and extracted with CH₂Cl₂. The combined organic phases were dried over MgSO₄, filtered, and concentrated. The crude material obtained was used directly without further purification. LC-MS calculated for C₁₄H₂₂N₅O₂ (M+H)⁺: m/z=292.2; found 292.1.

### Step 4: 7-(1-(1-Ethoxyethyl)-1H-pyrazol-4-yl)-8-isopropoxy-[1,2,4]triazolo[1,5-c]pyrimidin-2-amine

To a mixture of 6-(1-(1-ethoxyethyl)-1H-pyrazol-4-yl)-5-isopropoxypyrimidin-4-amine (Step 3) in 1,4-dioxane (100 mL) was added O-ethyl carbonisothiocyanatidate (9.37 mL, 79 mmol, Oakwood 002560) and the reaction mixture was purged with N₂ and stirred at 90° C. for 8 h. After cooling to r.t., the reaction mixture was concentrated in vacuo. To the crude residue was added a mixture of hydroxylamine hydrochloride (25.8 g, 371 mmol) and N-ethyl-N-isopropylpropan-2-amine (64.7 mL, 371 mmol) in MeOH/EtOH (1:1, 200 mL) and the reaction mixture was stirred at 60° C. for 30 min. After cooling to r.t., the reaction mixture was concentrated in vacuo and the residue was diluted with saturated aqueous NH₄Cl and extracted with CH₂Cl₂. The combined organic phases were dried over MgSO₄, filtered, and concentrated. The residue was purified by silica gel column chromatography. LC-MS calculated for C₁₅H₂₂N₇O₂ (M+H)⁺: m/z=332.2; found 332.1.

### Intermediate 2. 8-Isopropoxy-7-(1H-pyrazol-4-yl)-[1,2,4]triazolo[1,5-c]pyrimidin-2-amine

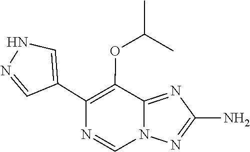 [SMILES: CC(C)Oc1c(-c2cn[nH]c2)ncn2nc(N)nc12]

To a mixture of 7-(1-(1-ethoxyethyl)-1H-pyrazol-4-yl)-8-isopropoxy-[1,2,4]triazolo[1,5-c]pyrimidin-2-amine (Intermediate 1, 0.40 g, 1.2 mmol) in CH₂Cl₂/MeOH (4:1, 5 mL) was added a 4 molar solution of HCl in 1,4-dioxane (2 mL, 8 mmol) followed by a few drops of H₂O. After stirring at r.t. for 30 min, Et₂O (20 mL) was added slowly to the reaction mixture. The solid precipitate was collected via filtration, washed with Et₂O, and dried under vacuum. The crude material obtained was used directly without further purification. LC-MS calculated for C₁₁H₁₄N₇O (M+H)⁺: m/z=260.1; found 260.1.

### Intermediate 3. 2-Bromo-7-(1-(1-ethoxyethyl)-1H-pyrazol-4-yl)-8-isopropoxy-[1,2,4]triazolo[1,5-a]pyridine

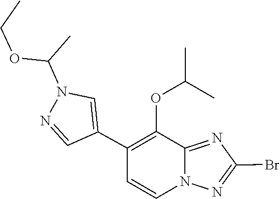 [SMILES: CCOC(C)n1cc(-c2ccn3nc(Br)nc3c2OC(C)C)cn1]

### Step 1: 4-Bromo-3-isopropoxypyridin-2-amine

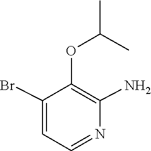

To a mixture of 2-amino-4-bromopyridin-3-ol hydrobromide (1.15 g, 4.26 mmol, eNovation D462032) in CH₃CN (20 mL) was added Cs₂CO₃ (2.78 g, 8.52 mmol) and the reaction mixture was stirred at 50° C. for 30 min before 2-iodopropane (0.64 mL, 6.4 mmol) was added and the reaction mixture was stirred at 70° C. overnight. After cooling to r.t., the reaction mixture was diluted with CH₂Cl₂ and filtered through Celite® and SiO₂. The filter cake was washed with CH₂Cl₂ and the filtrate was concentrated. The crude residue was purified by flash column chromatography (40 g SiO₂, EtOAc/hexanes) to afford the desired product. LC-MS calculated for C₈H₁₂BrN₂O (M+H)⁺: m/z=231.0; found 230.9.

### Step 2: 4-(1-(1-Ethoxyethyl)-1H-pyrazol-4-yl)-3-isopropoxypyridin-2-amine

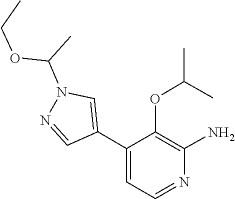 [SMILES: CCOC(C)n1cc(-c2ccnc(N)c2OC(C)C)cn1]

A mixture of 4-bromo-3-isopropoxypyridin-2-amine (0.70 g, 3.0 mmol), 1-(1-ethoxyethyl)-4-(4,4,5,5-tetramethyl-1,3,2-dioxaborolan-2-yl)-1H-pyrazole (1.2 g, 4.5 mmol, Combi-Blocks FM-3403), chloro(2-dicyclohexylphosphino-2′,4′,6′-triisopropyl-1,1′-biphenyl)[2-(2′-amino-1,1′-biphenyl)]palladium(II) (0.48 g, 0.61 mmol), K₃PO₄ (1.93 g, 9.1 mmol) in 1,4-dioxane (10 mL) and H₂O (2 mL) was purged with N₂ and stirred at 100° C. for 2 h. After cooling to r.t., the reaction mixture was diluted with H₂O and extracted with CH₂Cl₂. The combined organic phases were dried over MgSO₄, filtered, and concentrated. The crude residue was purified by flash column chromatography (40 g SiO₂, CH₂Cl₂/MeOH) to afford the desired product. LC-MS calculated for C₁₅H₂₃N₄O₂ (M+H)⁺: m/z=291.2; found 291.1.

### Step 3: 7-(1-(1-Ethoxyethyl)-1H-pyrazol-4-yl)-8-isopropoxy-[1,2,4]triazolo[1,5-a]pyridin-2-amine

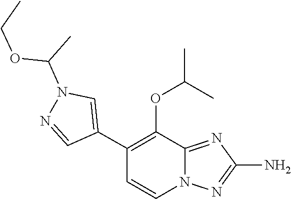 [SMILES: CCOC(C)n1cc(-c2ccn3nc(N)nc3c2OC(C)C)cn1]

A mixture of 4-(1-(1-ethoxyethyl)-1H-pyrazol-4-yl)-3-isopropoxypyridin-2-amine (0.90 g, 3.1 mmol), O-ethyl carbonisothiocyanatidate (0.52 mL, 4.34 mmol, Oakwood 002560) in 1,4-dioxane (15 mL) was purged with N₂ and stirred at 50° C. for 4 h. After cooling to r.t., the reaction mixture was concentrated. To the residue was added N-ethyl-N-isopropylpropan-2-amine (1.1 mL, 6.2 mmol), hydroxylamine hydrochloride (0.65 g, 9.3 mmol), MeOH (20 mL) and EtOH (20 mL) and the reaction mixture was stirred at 100° C. for 2 h. After cooling to r.t., the reaction mixture was concentrated and the residue was diluted with saturated aqueous NaHCO₃ and extracted with CH₂Cl₂. The combined organic phases were dried over MgSO₄, filtered, and concentrated. The crude residue was purified by flash column chromatography (40 g SiO₂, CH₂Cl₂/MeOH) to afford the desired product. LC-MS calculated for C₁₆H₂₃N₆O₂ (M+H)⁺: m/z=331.2; found 331.1.

### Step 4: 2-Bromo-7-(1-(1-ethoxyethyl)-1H-pyrazol-4-yl)-8-isopropoxy-[1,2,4]triazolo[1,5-a]pyridine

To a mixture of 7-(1-(1-ethoxyethyl)-1H-pyrazol-4-yl)-8-isopropoxy-[1,2,4]triazolo[1,5-a]pyridin-2-amine (0.50 g, 1.5 mmol), tert-butyl nitrite (90 wt %, 0.54 mL, 4.1 mmol) and CuBr₂ (0.44 g, 2.0 mmol) in CH₃CN (5 mL) was stirred at 50° C. for 1 h. After cooling to r.t., the reaction mixture was treated with a 35% aqueous ammonia solution, diluted with water, and extracted with CH₂Cl₂. The combined organic phases were dried over MgSO₄, filtered, and concentrated. The crude residue was purified by flash column chromatography (40 g SiO₂, EtOAc/hexanes) to afford the desired product. LC-MS calculated for C₁₆H₂₁BrN₅O₂(M+H)⁺: m/z=394.1; found 394.0.

### Intermediate 4. 7-(1-(1-Ethoxyethyl)-1H-pyrazol-4-yl)-8-((1,1,1-trifluoropropan-2-yl)oxy)-[1,2,4]triazolo[1,5-a]pyridin-2-amine

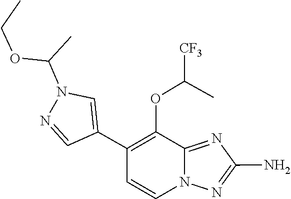 [SMILES: CCOC(C)n1cc(-c2ccn3nc(N)nc3c2OC(C)C(F)(F)F)cn1]

### Step 1: 4-Bromo-3-((1,1,1-trifluoropropan-2-yl)oxy)pyridin-2-amine

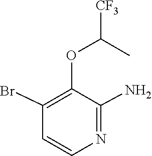

A mixture of 2-amino-4-bromopyridin-3-ol hydrobromide (8.00 g, 29.6 mmol, eNovation D462032) and Cs₂CO₃ (2.72 g, 8.34 mmol) in MeCN (9 mL) was stirred at 50° C. for 30 min before 1,1,1-trifluoropropan-2-yl trifluoromethanesulfonate (0.821 g, 3.33 mmol, Oakwood 102368) was added and the reaction mixture was stirred at 70° C. for 3 h. The reaction mixture was allowed to cool to r.t. overnight. The mixture was diluted with CH₂Cl₂ and filtered through Celite® and SiO₂. The filter cake was washed with CH₂Cl₂ and the filtrate was concentrated in vacuo. The crude residue was purified by flash column chromatography (SiO₂, EtOAc/hexanes) to afford the desired product (380 mg, 40% yield). LC-MS calculated for C₈H₉BrF₃N₂O (M+H)⁺: m/z=285.0; found 285.0.

### Step 2: 4-(1-(1-Ethoxyethyl)-1H-pyrazol-4-yl)-3-((1,1,1-trifluoropropan-2-yl)oxy)pyridin-2-amine

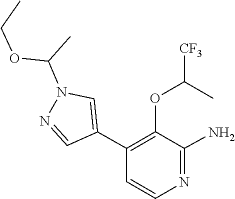 [SMILES: CCOC(C)n1cc(-c2ccnc(N)c2OC(C)C(F)(F)F)cn1]

A mixture of 4-bromo-3-((1,1,1-trifluoropropan-2-yl)oxy)pyridin-2-amine (0.40 g, 1.403 mmol), 1-(1-ethoxyethyl)-4-(4,4,5,5-tetramethyl-1,3,2-dioxaborolan-2-yl)-1H-pyrazole (0.560 g, 2.11 mmol, Combi-Blocks FM-3403), XPhos Pd G2 (221 mg, 0.281 mmol, Aldrich 741825), and K₃PO₄ (893 mg, 4.21 mmol) in 1,4-dioxane/H₂O (5:1, 6 mL) was stirred at 100° C. for 2 h. After cooling to r.t., the reaction mixture was diluted with H₂O and extracted with CH₂Cl₂. The combined organic phases were dried over MgSO₄ and concentrated in vacuo. The crude residue was purified by flash column chromatography (SiO₂, CH₂Cl₂/EtOAc) to afford the desired product (410 mg, 85% yield). LC-MS calculated for C₁₅H₂₀F₃N₄O₂(M+H)⁺: m/z=345.2; found 345.1.

### Step 3: 7-(1-(1-Ethoxyethyl)-1H-pyrazol-4-yl)-8-((1,1,1-trifluoropropan-2-yl)oxy)-[1,2,4]triazolo[1,5-a]pyridin-2-amine

To a mixture of 4-(1-(1-ethoxyethyl)-1H-pyrazol-4-yl)-3-((1,1,1-trifluoropropan-2-yl)oxy)pyridin-2-amine (410 mg, 1.19 mmol) in 1,4-dioxane (2.0 mL) was added O-ethyl carbonisothiocyanatidate (0.20 mL, 1.7 mmol, Oakwood 002560) and the reaction mixture was stirred at 50° C. for 2 h. The reaction mixture was concentrated in vacuo. The residue was taken up in MeOH/EtOH (1:1, 6 mL) and N-ethyl-N-isopropylpropan-2-amine (613 μL, 3.51 mmol) was added followed by hydroxylamine hydrochloride (0.195 g, 2.81 mmol) and the reaction mixture was stirred at 100° C. for 2 h. After cooling to r.t., the reaction mixture was concentrated in vacuo. The residue was taken up in CH₂Cl₂ and washed with saturated aqueous NaHCO₃, water, and brine successively. The organic layer was dried over MgSO₄, filtered, and concentrated in vacuo. The crude residue was purified by flash column chromatography (SiO₂, CH₂Cl₂/EtOAc) to afford the desired product. LC-MS calculated for C₁₆H₂₀F₃N₆O₂(M+H)⁺: m/z=385.2; found 385.2.

### Intermediate 5. 1,1-Difluoropropan-2-yl 1,1,2,2,3,3,4,4,4-nonafluorobutane-1-sulfonate

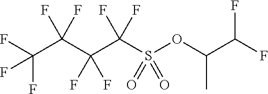 [SMILES: CC(OS(=O)(=O)C(F)(F)C(F)(F)C(F)(F)C(F)(F)F)C(F)F]

A mixture of 1,1-difluoropropan-2-ol (3.0 g, 31.2 mmol, Enamine EN300-170767) and N-ethyl-N-isopropylpropan-2-amine (5.45 mL, 31.2 mmol) in CH₂Cl₂ (15.6 mL) under N₂ was cooled to −40° C. before 1,1,2,2,3,3,4,4,4-nonafluorobutane-1-sulfonyl fluoride (9.43 g, 31.2 mmol) was added and the reaction mixture was allowed to warm to r.t. and stirred for 14 h. The mixture was diluted with CH₂Cl₂ and washed with saturated aqueous NaHCO₃. The organic layer was dried over MgSO₄, filtered, and concentrated in vacuo. The crude material obtained (11.5 g) was used directly without further purification.

### Intermediate 6. 8-((1,1-Difluoropropan-2-yl)oxy)-7-(1-(1-ethoxyethyl)-1H-pyrazol-4-yl)-[1,2,4]triazolo[1,5-a]pyridin-2-amine

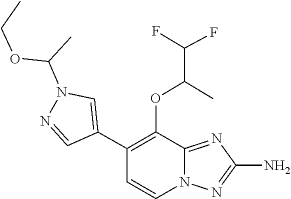 [SMILES: CCOC(C)n1cc(-c2ccn3nc(N)nc3c2OC(C)C(F)F)cn1]

Step 1: 4-Bromo-3-((1,1-difluoropropan-2-yl)oxy)pyridin-2-amine

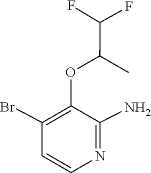

A mixture of 2-amino-4-bromopyridin-3-ol hydrobromide (8.00 g, 29.6 mmol, eNovation D462032) and Cs₂CO₃ (24.1 g, 74.0 mmol) in MeCN (70 mL) was stirred at 50° C. for 30 min before 1,1-difluoropropan-2-yl 1,1,2,2,3,3,4,4,4-nonafluorobutane-1-sulfonate (Intermediate 5, 11.2 g, 29.6 mmol) was added and the reaction mixture was stirred at 70° C. for 2 h. The reaction mixture was allowed to cool to r.t. overnight. The mixture was diluted with CH₂Cl₂ and filtered through Celite® and SiO₂. The filter cake was washed with CH₂Cl₂ and the filtrate was concentrated in vacuo. The crude residue was purified by flash column chromatography (40 g SiO₂, EtOAc/hexanes) to afford the desired product (2.25 g, 28% yield). LC-MS calculated for C₈H₁₀BrF₂N₂O (M+H)⁺: m/z=267.0; found 267.0.

### Step 2: 3-((1,1-Difluoropropan-2-yl)oxy)-4-(1-(I-ethoxyethyl)-1H-pyrazol-4-yl)pyridin-2-amine

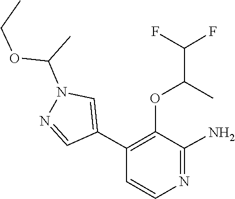 [SMILES: CCOC(C)n1cc(-c2ccnc(N)c2OC(C)C(F)F)cn1]

A mixture of 4-bromo-3-((1,1-difluoropropan-2-yl)oxy)pyridin-2-amine (2.25 g, 8.42 mmol), 1-(1-ethoxyethyl)-4-(4,4,5,5-tetramethyl-1,3,2-dioxaborolan-2-yl)-1H-pyrazole (3.36 g, 12.6 mmol, Combi-Blocks FM-3403), XPhos Pd G2 (1.33 g, 1.69 mmol, Aldrich 741825), and K₃PO₄ (5.36 g, 25.3 mmol) in 1,4-dioxane/H₂O (5:1, 42 mL) was stirred at 100° C. for 2 h. After cooling to r.t., the reaction mixture was diluted with H₂O and extracted with CH₂Cl₂. The combined organic phases were dried over MgSO₄ and concentrated in vacuo. The crude residue was purified by flash column chromatography (20 g SiO₂, CH₂Cl₂/EtOAc) to afford the desired product (2.50 g, 91% yield). LC-MS calculated for C₁₅H₂₁F₂N₄O₂(M+H)⁺: m/z=327.2; found 327.1.

### Step 3: 8-((1,1-Difluoropropan-2-yl)oxy)-7-(1-(I-ethoxyethyl)-1H-pyrazol-4-yl)-[1,2,4]triazolo[1,5-a]pyridin-2-amine

To a mixture of 3-((1,1-difluoropropan-2-yl)oxy)-4-(1-(1-ethoxyethyl)-1H-pyrazol-4-yl)pyridin-2-amine (2.50 g, 7.66 mmol) in 1,4-dioxane (20.0 mL) was added O-ethyl carbonisothiocyanatidate (1.21 mL, 10.1 mmol, Oakwood 002560) and the reaction mixture was stirred at 50° C. for 2 h. The reaction mixture was concentrated in vacuo. The residue was taken up in MeOH/EtOH (1:1, 30 mL) and N-ethyl-N-isopropylpropan-2-amine (3.68 mL, 21.1 mmol) was added followed by hydroxylamine hydrochloride (1.17 g, 16.8 mmol) and the reaction mixture was stirred at 100° C. for 2 h. After cooling to r.t., the reaction mixture was concentrated in vacuo. The residue was taken up in CH₂Cl₂ and washed with saturated aqueous NaHCO₃, water, and brine successively. The organic layer was dried over MgSO₄, filtered, and concentrated in vacuo. The crude residue was purified by silica gel flash column chromatography to afford the desired product (1.17 g, 42% yield). LC-MS calculated for C₁₆H₂₁F₂N₆O₂(M+H)⁺: m/z=367.2; found 367.2.

### Intermediate 7. 7-(1-(1-Ethoxyethyl)-1H-pyrazol-4-yl)-8-((1,1,1-trifluoropropan-2-yl)oxy)-[1,2,4]triazolo[1,5-c]pyrimidin-2-amine

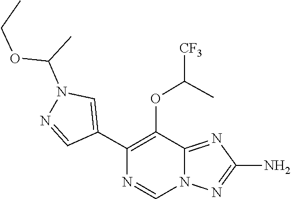 [SMILES: CCOC(C)n1cc(-c2ncn3nc(N)nc3c2OC(C)C(F)(F)F)cn1]

### Step 1: 6-Chloro-5-((1,1,1-trifluoropropan-2-yl)oxy)pyrimidin-4-amine

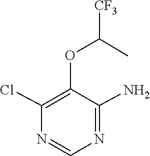

A mixture of 4-amino-6-chloropyrimidin-5-ol (3.25 g, 22.3 mmol, BLD Pharmatech BD166906) and Cs₂CO₃ (10.9 g, 33.5 mmol) in MeCN (40 mL) was stirred at 50° C. for 30 min before 1,1,1-trifluoropropan-2-yl trifluoromethanesulfonate (6.60 g, 26.8 mmol, Oakwood 102368) was added and the reaction mixture was stirred at 70° C. for 4 h. The reaction mixture was allowed to cool to r.t. overnight. The mixture was diluted with water and extracted with EtOAc. The combined organic phases were dried over MgSO₄ and concentrated in vacuo. The crude residue was purified by flash column chromatography (20 g SiO₂, EtOAc/hexanes) to afford the desired product (2.4 g, 44% yield). LC-MS calculated for C₇H₈ClF₃N₃O (M+H)⁺: m/z=242.0; found 242.1.

### Step 2: 6-(1-(1-Ethoxyethyl)-1H-pyrazol-4-yl)-5-((1,1,1-trifluoropropan-2-yl)oxy)pyrimidin-4-amine

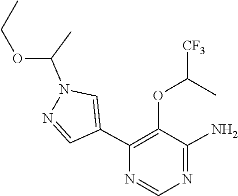 [SMILES: CCOC(C)n1cc(-c2ncnc(N)c2OC(C)C(F)(F)F)cn1]

A mixture of 6-chloro-5-((1,1,1-trifluoropropan-2-yl)oxy)pyrimidin-4-amine (2.4 g, 9.9 mmol), 1-(1-ethoxyethyl)-4-(4,4,5,5-tetramethyl-1,3,2-dioxaborolan-2-yl)-1H-pyrazole (3.97 g, 14.9 mmol, Combi-Blocks FM-3403), XPhos Pd G2 (1.56 g, 1.99 mmol, Aldrich 741825), and K₃PO₄ (6.32 g, 29.8 mmol) in 1,4-dioxane/H₂O (5:1, 42 mL) was stirred at 100° C. for 2 h. After cooling to r.t., the reaction mixture was diluted with H₂O and extracted with CH₂Cl₂. The combined organic phases were dried over MgSO₄ and concentrated in vacuo. The crude residue was purified by flash column chromatography (20 g SiO₂, CH₂Cl₂/EtOAc) to afford the desired product (2.64 g, 77% yield). LC-MS calculated for C₁₄H₁₉F₃N₅O₂(M+H)⁺: m/z=346.1; found 346.2.

### Step 3: 7-(1-(1-Ethoxyethyl)-1H-pyrazol-4-yl)-8-((1,1,1-trifluoropropan-2-yl)oxy)-[1,2,4]triazolo[1,5-c]pyrimidin-2-amine

To a mixture of 6-(1-(1-ethoxyethyl)-1H-pyrazol-4-yl)-5-((1,1,1-trifluoropropan-2-yl)oxy)pyrimidin-4-amine (2.64 g, 7.64 mmol) in 1,4-dioxane (40 mL) was added O-ethyl carbonisothiocyanatidate (1.78 mL, 14.9 mmol, Oakwood 002560) and the reaction mixture was stirred at 100° C. for 4.5 h. The reaction mixture was concentrated in vacuo. The residue was taken up in MeOH/EtOH (1:1, 30 mL) and N-ethyl-N-isopropylpropan-2-amine (6.94 mL, 39.7 mmol) was added followed by hydroxylamine hydrochloride (1.381 g, 19.87 mmol) and the reaction mixture was stirred at 100° C. for 1 h. After cooling to r.t., the reaction mixture was concentrated in vacuo. The residue was taken up in CH₂Cl₂ and washed with saturated aqueous NaHCO₃, water, and brine successively. The organic layer was dried over MgSO₄, filtered, and concentrated in vacuo. The crude residue was purified by silica gel flash column chromatography to afford the desired product. LC-MS calculated for C₁₅H₁₉F₃N₇O₂(M+H)⁺: m/z=386.2; found 386.1.

### Intermediate 8. 8-((1,1-Difluoropropan-2-yl)oxy)-7-(1-(1-ethoxyethyl)-1H-pyrazol-4-yl)-[1,2,4]triazolo[1,5-c]pyrimidin-2-amine

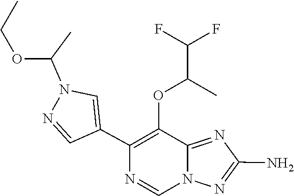 [SMILES: CCOC(C)n1cc(-c2ncn3nc(N)nc3c2OC(C)C(F)F)cn1]

### Step 1: 6-Chloro-5-((1,1-difluoropropan-2-yl)oxy)pyrimidin-4-amine

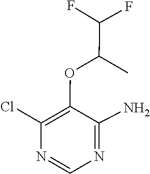

A mixture of 4-amino-6-chloropyrimidin-5-ol (6.00 g, 41.2 mmol, BLD Pharmatech BD166906) and Cs₂CO₃ (20.2 g, 62 mmol) in MeCN (70 mL) was stirred at 50° C. for 30 min before 1,1-difluoropropan-2-yl 1,1,2,2,3,3,4,4,4-nonafluorobutane-1-sulfonate (Intermediate 5, 15.6 g, 41.3 mmol) was added and the reaction mixture was stirred at 70° C. for 3 h. The reaction mixture was allowed to cool to r.t. overnight. The mixture was diluted with CH₂Cl₂ and filtered through Celite® and SiO₂. The filter cake was washed with CH₂Cl₂ and the filtrate was concentrated in vacuo. The crude residue was purified by flash column chromatography (40 g SiO₂, EtOAc/hexanes) to afford the desired product (2.90 g, 31% yield). LC-MS calculated for C₇H₉ClF₂N₃O (M+H)⁺: m/z=224.0; found 224.1.

### Step 2: 5-((1,1-Difluoropropan-2-yl)oxy)-6-(1-(I-ethoxyethyl)-1H-pyrazol-4-yl)pyrimidin-4-amine

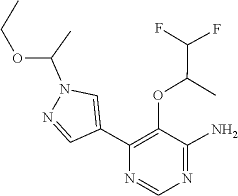 [SMILES: CCOC(C)n1cc(-c2ncnc(N)c2OC(C)C(F)F)cn1]

A mixture of 6-chloro-5-((1,1-difluoropropan-2-yl)oxy)pyrimidin-4-amine (2.90 g, 13.0 mmol), 1-(1-ethoxyethyl)-4-(4,4,5,5-tetramethyl-1,3,2-dioxaborolan-2-yl)-1H-pyrazole (5.18 g, 19.5 mmol, Combi-Blocks FM-3403), XPhos Pd G2 (2.04 g, 2.59 mmol, Aldrich 741825), and K₃PO₄ (8.26 g, 38.9 mmol) in 1,4-dioxane/H₂O (5:1, 42 mL) was stirred at 70° C. for 2 h. After cooling to r.t., the reaction mixture was diluted with H₂O and extracted with CH₂Cl₂. The combined organic phases were dried over MgSO₄ and concentrated in vacuo. The crude residue was purified by flash column chromatography (20 g SiO₂, CH₂Cl₂/EtOAc) to afford the desired product (1.20 g, 28% yield). LC-MS calculated for C₁₄H₂₀F₂N₅O₂(M+H)⁺: m/z=328.2; found 328.2.

### Step 3: 8-((1,1-Difluoropropan-2-yl)oxy)-7-(1-(I-ethoxyethyl)-1H-pyrazol-4-yl)-[1,2,4]triazolo[1,5-c]pyrimidin-2-amine

To a mixture of 5-((1,1-difluoropropan-2-yl)oxy)-6-(1-(1-ethoxyethyl)-1H-pyrazol-4-yl)pyrimidin-4-amine (1.20 g, 3.67 mmol) in 1,4-dioxane (20.0 mL) was added O-ethyl carbonisothiocyanatidate (1.55 mL, 13.0 mmol) and the reaction mixture was stirred at 90° C. for 4 h. The reaction mixture was concentrated in vacuo. The residue was taken up in MeOH/EtOH (1:1, 30 mL) and N-ethyl-N-isopropylpropan-2-amine (4.53 mL, 25.9 mmol) was added followed by hydroxylamine hydrochloride (1.35 g, 19.4 mmol) and the reaction mixture was stirred at 70° C. for 2 h. After cooling to r.t., the reaction mixture was concentrated in vacuo. The residue was taken up in CH₂Cl₂ and washed with saturated aqueous NaHCO₃, water, and brine successively. The organic layer was dried over MgSO₄, filtered, and concentrated in vacuo. The crude residue was purified by silica gel flash column chromatography to afford the desired product (430 mg, 32% yield). LC-MS calculated for C₁₅H₂₀F₂N₇O₂(M+H)⁺: m/z=368.2; found 368.2.

### Example 1. 8-Isopropoxy-7-(1H-pyrazol-4-yl)-N-(tetrahydro-2H-pyran-4-yl)-[1,2,4]triazolo[1,5-c]pyrimidin-2-amine

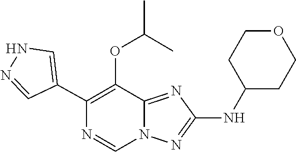 [SMILES: CC(C)Oc1c(-c2cn[nH]c2)ncn2nc(NC3CCOCC3)nc12]

A mixture of sodium triacetoxyborohydride (3.19 g, 15.0 mmol) in TFA (20 mL) was added dropwise to a mixture of 8-isopropoxy-7-(1H-pyrazol-4-yl)-[1,2,4]triazolo[1,5-c]pyrimidin-2-amine (Intermediate 2, 1.30 g, 5.01 mmol) and tetrahydro-4H-pyran-4-one (1.506 g, 15.0 mmol) in DMF/TFA (1:1, 40 mL) and the reaction mixture was stirred at ambient temperature for 30 min. The reaction mixture was concentrated to half volume and diluted with EtOAc (100 mL). The mixture was diluted with saturated aqueous NaHCO₃ until the aqueous layer reached alkaline pH and extracted with EtOAc (3×200 mL). The combined organic layers were dried over MgSO₄, concentrated, and the crude residue was purified by flash column chromatography (SiO₂, 0-5% MeOH/CH₂Cl₂) to afford the desired product (0.65 g, 38% yield) as a light yellow solid. To a mixture of the product in MeCN/H₂O (1:1, 50 mL) was added TFA (0.3 mL) and the resulting mixture was lyophilized to afford the desired product as its TFA salt. LC-MS calculated for C₁₆H₂₂N₇O₂ (M+H)⁺: m/z=344.2; found 344.1. ¹H NMR (500 MHz, DMSO-d₆) δ 9.11 (s, 1H), 8.23 (s, 2H), 7.24-6.87 (brs, 1H), 5.55 (hept, J=6.2 Hz, 1H), 3.91-3.84 (m, 2H), 3.73-3.63 (m, 1H), 3.43-3.35 (m, 2H), 1.96-1.88 (m, 2H), 1.57-1.46 (m, 2H), 1.31 (d, J=6.2 Hz, 6H).

### Example 2. 8-Isopropoxy-7-(1H-pyrazol-4-yl)-N-(tetrahydro-2H-pyran-4-yl)-[1,2,4]triazolo[1,5-a]pyridin-2-amine

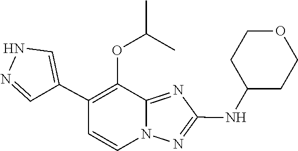 [SMILES: CC(C)Oc1c(-c2cn[nH]c2)ccn2nc(NC3CCOCC3)nc12]

### Step 1: 7-(1-(1-Ethoxyethyl)-1H-pyrazol-4-yl)-8-isopropoxy-N-(tetrahydro-2H-pyran-4-yl)-[1,2,4]triazolo[1,5-a]pyridin-2-amine

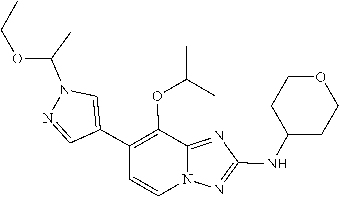 [SMILES: CCOC(C)n1cc(-c2ccn3nc(NC4CCOCC4)nc3c2OC(C)C)cn1]

To a mixture of 2-bromo-7-(1-(1-ethoxyethyl)-1H-pyrazol-4-yl)-8-isopropoxy-[1,2,4]triazolo[1,5-a]pyridine (Intermediate 3, 30.0 mg, 0.076 mmol) in 1,4-dioxane (0.5 mL) was added tetrahydro-2H-pyran-4-amine (15.0 mg, 0.152 mmol), methanesulfonato[2-(di-1-adamantylphosphino)-3,6-dimethoxy-2′,4′,6′-tri-i-propyl-1,1′-biphenyl](2′-amino-1,1′-biphenyl-2-yl)palladium(II) (26 mg, 0.025 mmol, Strem 46-0480), and sodium tert-butoxide (29.0 mg, 0.304 mmol). The reaction mixture was purged with N₂ and stirred at 120° C. for 1 h. After cooling to r.t., the reaction mixture was diluted with CH₂Cl₂ (5 mL) and filtered through a pad of MgSO₄ in a SiliaPrep SPE thiol cartridge (500 mg, SiliCycle SPE-R51030B-06P). The filtrate was concentrated, and the crude residue material obtained was used directly without purification. LC-MS calculated for C₂₁H₃₁N₆O₃ (M+H)⁺: m/z=415.2; found 415.3.

### Step 2: 8-Isopropoxy-7-(1H-pyrazol-4-yl)-N-(tetrahydro-2H-pyran-4-yl)-[1,2,4]triazolo[1,5-a]pyridin-2-amine

To a mixture of 7-(1-(1-ethoxyethyl)-1H-pyrazol-4-yl)-8-isopropoxy-N-(tetrahydro-2H-pyran-4-yl)-[1,2,4]triazolo[1,5-a]pyridin-2-amine (Step 1) in MeOH (0.5 mL) was added a 4 molar solution of HCl in 1,4-dioxane (380 μL, 1.52 mmol) and the reaction mixture was stirred at r.t. for 30 min. The mixture was diluted with CH₃CN and H₂O, filtered, and purified by prep-HPLC (Sunfire C18 column, eluting with a gradient of acetonitrile/water containing 0.1% TFA, at flow rate of 60 mL/min) to afford the desired product as its TFA salt. LC-MS calculated for C₁₇H₂₃N₆O₂ (M+H)⁺: m/z=343.2; found 343.1. ¹H NMR (600 MHz, DMSO-d₆) δ 8.32 (d, J=7.0 Hz, 1H), 8.24 (s, 2H), 7.18 (d, J=7.0 Hz, 1H), 5.53 (hept, J=6.1 Hz, 1H), 3.90-3.84 (m, 2H), 3.69-3.61 (m, 1H), 3.42-3.35 (m, 2H), 1.95-1.89 (m, 2H), 1.55-1.46 (m, 2H), 1.25 (d, J=6.1 Hz, 6H).

### Examples 3 and 4. (R)-7-(1H-Pyrazol-4-yl)-N-(tetrahydro-2H-pyran-4-yl)-8-((1,1,1-trifluoropropan-2-yl)oxy)-[1,2,4]triazolo[1,5-a]pyridin-2-amine and (S)-7-(1H-pyrazol-4-yl)-N-(tetrahydro-2H-pyran-4-yl)-8-((1,1,1-trifluoropropan-2-yl)oxy)-[1,2,4]triazolo[1,5-a]pyridin-2-amine

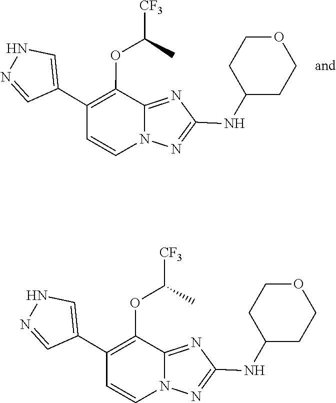 [SMILES: C[C@@H](Oc1c(-c2cn[nH]c2)ccn2nc(NC3CCOCC3)nc12)C(F)(F)F ; C[C@H](Oc1c(-c2cn[nH]c2)ccn2nc(NC3CCOCC3)nc12)C(F)(F)F]

A mixture of 7-(1-(1-ethoxyethyl)-1H-pyrazol-4-yl)-8-((1,1,1-trifluoropropan-2-yl)oxy)-[1,2,4]triazolo[1,5-a]pyridin-2-amine (Intermediate 4, 460 mg, 1.20 mmol) and tetrahydro-4H-pyran-4-one (0.331 mL, 3.59 mmol) in DMF/TFA (1:1, 4 mL) was stirred at r.t. for 30 min. A mixture of sodium triacetoxyborohydride (1.015 g, 4.79 mmol) in TFA (2 mL) was added dropwise and the reaction mixture was stirred at ambient temperature overnight. The reaction mixture was diluted with MeOH (20 mL) and concentrated in vacuo. The crude residue was diluted once more with MeOH (20 mL) and the concentrated in vacuo. The crude residue was taken up in CH₂Cl₂ (2 mL) and triethylamine (0.500 mL, 3.59 mmol) was added. The mixture was purified directly by flash column chromatography (SiO₂, 0-10% MeOH in CH₂Cl₂) to afford the desired product as a racemic mixture. The racemic mixture was purified by preparative chiral SFC (Phenomenex Lux 5 μm i-cellulose-5, 2.21×250 mm, eluting with an isocratic mobile phase of 12% MeOH with 5 mM NH₃ in CO₂, at flow rate of 70 mL/min). Each fraction was concentrated in vacuo. The residue was taken up in MeCN/H₂O (1:1, 10 mL) and the mixture was lyophylized to afford each enantiomer.

Example 3: Retention time on preparative chiral HPLC tᵣ=11.5 min, LC-MS calculated for C₁₇H₂₀F₃N₆O₂(M+H)⁺: m/z=397.2; found 397.1. ¹H NMR (600 MHz, DMSO-d₆) δ 13.21-13.08 (brs, 1H), 8.40 (d, J=7.0 Hz, 1H), 8.34-8.23 (brs, 1H), 8.16-8.05 (brs, 1H), 7.22 (d, J=7.0 Hz, 1H), 6.72 (d, J=7.6 Hz, 1H), 6.17 (hept, J=6.7 Hz, 1H), 3.91-3.85 (m, 2H), 3.70-3.61 (m, 1H), 3.42-3.35 (m, 2H), 1.96-1.88 (m, 2H), 1.56-1.48 (m, 2H), 1.47 (d, J=6.5 Hz, 3H).

Example 4: Retention time on preparative chiral HPLC tᵣ=12.75 min, LC-MS calculated for C₁₇H₂₀F₃N₆O₂(M+H)⁺: m/z=397.2; found 397.1. ¹H NMR (600 MHz, DMSO-d₆) δ 13.21-13.08 (brs, 1H), 8.40 (d, J=7.0 Hz, 1H), 8.34-8.22 (brs, 1H), 8.16-8.05 (brs, 1H), 7.22 (d, J=7.0 Hz, 1H), 6.72 (d, J=7.6 Hz, 1H), 6.17 (hept, J=6.7 Hz, 1H), 3.91-3.85 (m, 2H), 3.70-3.62 (m, 1H), 3.42-3.35 (m, 2H), 1.96-1.88 (m, 2H), 1.56-1.47 (m, 2H), 1.47 (d, J=6.5 Hz, 3H).

### Examples 5 and 6. (R)-8-((1,1-Difluoropropan-2-yl)oxy)-7-(1H-pyrazol-4-yl)-N-(tetrahydro-2H-pyran-4-yl)-[1,2,4]triazolo[1,5-a]pyridin-2-amine and (S)-8-((1,1-difluoropropan-2-yl)oxy)-7-(1H-pyrazol-4-yl)-N-(tetrahydro-2H-pyran-4-yl)-[1,2,4]triazolo[1,5-a]pyridin-2-amine

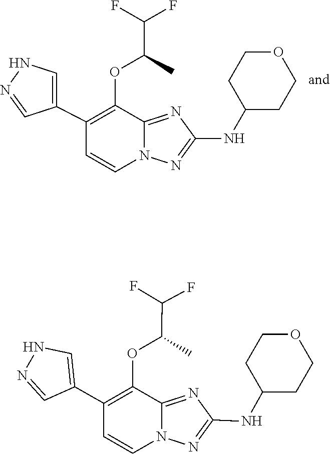 [SMILES: C[C@@H](Oc1c(-c2cn[nH]c2)ccn2nc(NC3CCOCC3)nc12)C(F)F ; C[C@H](Oc1c(-c2cn[nH]c2)ccn2nc(NC3CCOCC3)nc12)C(F)F]

A mixture of 8-((1,1-difluoropropan-2-yl)oxy)-7-(1-(1-ethoxyethyl)-1H-pyrazol-4-yl)-[1,2,4]triazolo[1,5-a]pyridin-2-amine (Intermediate 6, 250 mg, 0.682 mmol) and tetrahydro-4H-pyran-4-one (0.189 mL, 2.05 mmol) in DMF/TFA (1:1, 4 mL) was stirred at r.t. for 30 min. A mixture of sodium triacetoxyborohydride (578 mg, 2.73 mmol) in TFA (2 mL) was added dropwise and the reaction mixture was stirred at ambient temperature overnight. The reaction mixture was diluted with MeOH (20 mL) and concentrated in vacuo. The crude residue was diluted once more with MeOH (20 mL) and the concentrated in vacuo. The crude residue was taken up in CH₂Cl₂ (2 mL) and triethylamine (0.285 mL, 2.05 mmol) was added. The mixture was purified directly by flash column chromatography (SiO₂, 0-10% MeOH in CH₂Cl₂) to afford the desired product as a racemic mixture. The racemic mixture was purified by preparative chiral SFC (ChiralTech IK 5 μm, 21×250 mm, eluting with a mobile phase of 20% MeOH in CO₂, at flow rate of 70 mL/min). Each fraction was concentrated in vacuo. The residue was taken up in MeCN/H₂O (1:1, 10 mL) and the mixture was lyophylized to afford each enantiomer. Further purification by prep-HPLC (Sunfire C18 column, eluting with a gradient of acetonitrile/water containing 0.1% TFA, at flow rate of 60 mL/min) afforded each desired product as its TFA salt.

Example 5: Retention time on preparative chiral HPLC tᵣ=6.2 min, LC-MS calculated for C₁₇H₂₁F₂N₆O₂(M+H)+: m/z=379.2; found 379.1. ¹H NMR (500 MHz, DMSO-d₆) δ 8.36 (d, J=7.0 Hz, 1H), 8.23 (s, 2H), 7.21 (d, J=7.0 Hz, 1H), 6.49-6.21 (m, 1H), 5.85-5.72 (m, 1H), 3.91-3.84 (m, 2H), 3.70-3.61 (m, 1H), 3.42-3.34 (m, 2H), 1.96-1.88 (m, 2H), 1.57-1.45 (m, 2H), 1.31 (d, J=6.5 Hz, 3H).

Example 6: Retention time on preparative chiral HPLC tᵣ=7.0 min, LC-MS calculated for C₁₇H₂₁F₂N₆O₂(M+H)+: m/z=379.2; found 379.1. ¹H NMR (500 MHz, DMSO-d₆) δ 8.36 (d, J=7.0 Hz, 1H), 8.23 (s, 2H), 7.21 (d, J=7.0 Hz, 1H), 6.49-6.21 (m, 1H), 5.85-5.71 (m, 1H), 3.91-3.84 (m, 2H), 3.70-3.61 (m, 1H), 3.42-3.34 (m, 2H), 1.96-1.88 (m, 2H), 1.57-1.45 (m, 2H), 1.31 (d, J=6.5 Hz, 3H).

### Examples 7 and 8. (R)-7-(1H-Pyrazol-4-yl)-N-(tetrahydro-2H-pyran-4-yl)-8-((1,1,1-trifluoropropan-2-yl)oxy)-[1,2,4]triazolo[1,5-c]pyrimidin-2-amine and (S)-7-(1H-pyrazol-4-yl)-N-(tetrahydro-2H-pyran-4-yl)-8-((1,1,1-trifluoropropan-2-yl)oxy)-[1,2,4]triazolo[1,5-c]pyrimidin-2-amine

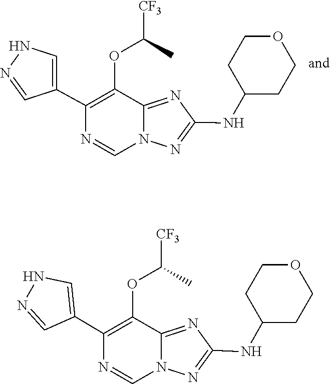 [SMILES: C[C@@H](Oc1c(-c2cn[nH]c2)ncn2nc(NC3CCOCC3)nc12)C(F)(F)F ; C[C@H](Oc1c(-c2cn[nH]c2)ncn2nc(NC3CCOCC3)nc12)C(F)(F)F]

A mixture of 7-(1-(1-ethoxyethyl)-1H-pyrazol-4-yl)-8-((1,1,1-trifluoropropan-2-yl)oxy)-[1,2,4]triazolo[1,5-c]pyrimidin-2-amine (Intermediate 7, 250 mg, 0.649 mmol) and tetrahydro-4H-pyran-4-one (0.180 mL, 1.95 mmol) in DMF/TFA (1:1, 4 mL) was stirred at r.t. for 30 min. A mixture of sodium triacetoxyborohydride (550 mg, 2.60 mmol) in TFA (2 mL) was added dropwise and the reaction mixture was stirred at ambient temperature overnight. The reaction mixture was diluted with MeOH (20 mL) and concentrated in vacuo. The crude residue was diluted once more with MeOH (20 mL) and the concentrated in vacuo. The crude residue was taken up in CH₂Cl₂ (2 mL) and triethylamine (0.271 mL, 1.95 mmol) was added. The mixture was purified directly by flash column chromatography (SiO₂, 0-10% MeOH in CH₂Cl₂) to afford the desired product as a racemic mixture. The racemic mixture was purified by preparative chiral HPLC (Chiral Tech IB—N 5 μm, 21×250 mm, eluting with an isocratic mobile phase of 10% EtOH in hexanes, at flow rate of 20 mL/min). Each fraction was concentrated in vacuo. The residue was taken up in MeCN/H₂O (1:1, 10 mL) and the mixture was lyophylized to afford each enantiomer.

Example 7: Retention time on preparative chiral HPLC tᵣ=12.1 min, LC-MS calculated for C₁₆H₁₉F₃N₇O₂(M+H)+: m/z=398.2; found 398.1. ¹H NMR (500 MHz, DMSO-d₆) δ 13.32-13.10 (brs, 1H), 9.19 (s, 1H), 8.41-7.97 (m, 2H), 7.18 (d, J=7.6 Hz, 1H), 6.05 (hept, J=6.6 Hz, 1H), 3.92-3.84 (m, 2H), 3.74-3.64 (m, 1H), 3.43-3.35 (m, 2H), 1.97-1.88 (m, 2H), 1.58-1.48 (m, 5H).

Example 8: Retention time on preparative chiral HPLC tᵣ=13.3 min, LC-MS calculated for C₁₆H₁₉F₃N₇O₂(M+H)+: m/z=398.2; found 398.1. ¹H NMR (600 MHz, DMSO-d₆) δ 13.32-13.10 (brs, 1H), 9.19 (s, 1H), 8.40-7.97 (m, 2H), 7.19 (d, J=7.6 Hz, 1H), 6.05 (hept, J=6.6 Hz, 1H), 3.92-3.84 (m, 2H), 3.73-3.64 (m, 1H), 3.42-3.35 (m, 2H), 1.96-1.88 (m, 2H), 1.58-1.48 (m, 5H).

### Examples 9 and 10. (R)-8-((1,1-Difluoropropan-2-yl)oxy)-7-(1H-pyrazol-4-yl)-N-(tetrahydro-2H-pyran-4-yl)-[1,2,4]triazolo[1,5-c]pyrimidin-2-amine and (S)-8-((1,1-difluoropropan-2-yl)oxy)-7-(1H-pyrazol-4-yl)-N-(tetrahydro-2H-pyran-4-yl)-[1,2,4]triazolo[1,5-c]pyrimidin-2-amine

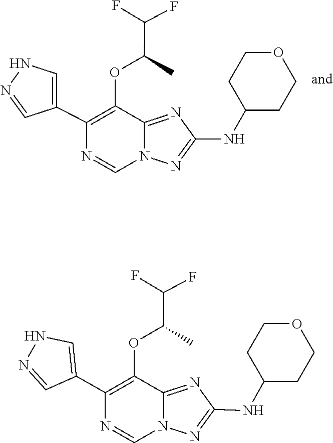 [SMILES: C[C@@H](Oc1c(-c2cn[nH]c2)ncn2nc(NC3CCOCC3)nc12)C(F)F ; C[C@H](Oc1c(-c2cn[nH]c2)ncn2nc(NC3CCOCC3)nc12)C(F)F]

A mixture of 8-((1,1-difluoropropan-2-yl)oxy)-7-(1-(1-ethoxyethyl)-1H-pyrazol-4-yl)-[1,2,4]triazolo[1,5-c]pyrimidin-2-amine (Intermediate 8, 320 mg, 0.871 mmol) and tetrahydro-4H-pyran-4-one (0.241 mL, 2.61 mmol) in DMF/TFA (1:1, 4 mL) was stirred at r.t. for 30 min. A mixture of sodium triacetoxyborohydride (738 mg, 3.48 mmol) in TFA (2 mL) was added dropwise and the reaction mixture was stirred at ambient temperature overnight. The reaction mixture was diluted with MeOH (20 mL) and concentrated in vacuo. The crude residue was diluted once more with MeOH (20 mL) and the concentrated in vacuo. The crude residue was taken up in CH₂Cl₂ (2 mL) and triethylamine (0.364 mL, 2.61 mmol) was added. The mixture was purified directly by flash column chromatography (SiO₂, 0-10% MeOH in CH₂Cl₂) to afford the desired product as a racemic mixture. The racemic mixture was purified by preparative chiral HPLC (Phenomenex Lux 5 μm i-Amylose-1, 21.2×250 mm, eluting with an isocratic mobile phase of 10% EtOH in hexanes, at flow rate of 20 mL/min). Each fraction was concentrated in vacuo. The residue was taken up in MeCN/H₂O (1:1, 10 mL) and the mixture was lyophylized to afford each enantiomer.

Example 9: Retention time on preparative chiral HPLC tᵣ=28.4 min, LC-MS calculated for C₁₆H₂₀F₂N₇O₂(M+H)+: m/z=380.2; found 380.1. ¹H NMR (600 MHz, DMSO-d₆) δ 13.29-13.04 (brs, 1H), 9.15 (s, 1H), 8.40-8.06 (m, 2H), 7.12 (d, J=7.6 Hz, 1H), 6.51-6.28 (m, 1H), 5.77-5.67 (m, 1H), 3.91-3.85 (m, 2H), 3.73-3.65 (m, 1H), 3.42-3.36 (m, 2H), 1.96-1.89 (m, 2H), 1.57-1.48 (m, 2H), 1.37 (d, J=6.6 Hz, 3H).

Example 10: Retention time on preparative chiral HPLC tᵣ=32.5 min, LC-MS calculated for C₁₆H₂₀F₂N₇O₂(M+H)+: m/z=380.2; found 380.1. ¹H NMR (600 MHz, DMSO-d₆) δ 13.29-13.03 (brs, 1H), 9.15 (s, 1H), 8.39-8.05 (m, 1H), 7.12 (d, J=7.6 Hz, 1H), 6.50-6.28 (m, 1H), 5.77-5.67 (m, 1H), 3.91-3.85 (m, 2H), 3.73-3.65 (m, 1H), 3.42-3.36 (m, 2H), 1.96-1.89 (m, 2H), 1.57-1.48 (m, 3H), 1.37 (d, J=6.6 Hz, 3H).

### Example A. CDK2/Cyclin E1 HTRF Enzyme Activity Assay

CDK2/Cyclin E1 enzyme activity assays utilize full-length human CDK2 co-expressed as N-terminal GST-tagged protein with FLAG-Cyclin E1 in a baculovirus expression system (Carna Product Number 04-165). Assays were conducted in white 384-well polystyrene plates in a final reaction volume of 8 μL. CDK2/Cyclin E1 (0.25 nM) was incubated with the compounds of the Examples (40 nL serially diluted in DMSO) in the presence of ATP (50 μM or 1 mM) and 50 nM ULight™-labeled eIF4E-binding protein 1 (THR37/46) peptide (PerkinElmer) in assay buffer (containing 50 mM HEPES pH 7.5, 1 mM EGTA, 10 mM MgCl₂, 2 mM DTT, 0.05 mg/mL BSA, and 0.01% Tween 20) for 60 minutes at room temperature. The reactions were stopped by the addition of EDTA and Europium-labeled anti-phospho-4E-BP1 antibody (PerkinElmer), for a final concentration of 15 mM and 1.5 nM, respectively. HTRF signals were read after 1 hour at room temperature on a PHERAstar FS plate reader (BMG Labtech). Data was analyzed with Genedata Screener software using a three or four parameter dose response curve to determine IC₅₀ for each compound. The IC₅₀ data as measured for the compounds of the Examples at 1 mM ATP in the assay of Example A is shown in Table 1.

TABLE 1

| Example | IC₅₀ (nM) |
|---|---|
| 1 | + |
| 2 | + |
| 3 | + |
| 4 | + |
| 5 | + |
| 6 | + |
| 7 | + |
| 8 | + |
| 9 | + |
| 10 | + |

+ refers to ≤10 nM

### Example B. In Vivo Rat CNS Protocol

The brain, CSF, and plasma concentrations of the compound of Example 1 (8-isopropoxy-7-(1H-pyrazol-4-yl)-N-(tetrahydro-2H-pyran-4-yl)-[1,2,4]triazolo[1,5-c]pyrimidin-2-amine) were determined in rats after IV dosing. The compound of Example 1 was formulated in 10% dimethylacetamide in acidified saline. Male rats (n=4) were given an IV bolus of the compound of Example 1 at 2.0 mg/kg, followed by an IV infusion at 1.2 mg/kg/h for 4 hours. Blood samples were collected at 2 hours and 4 hours in tubes containing EDTA as anticoagulant and centrifuged to obtain plasma. At 4 hours, brain and CSF samples were also collected. The CSF samples were mixed with blank rat plasma (1:1 v/v) to minimize adsorption of the compound of Example 1 to the collection device. Brain, CSF, and plasma samples were frozen and concentrations of the compound of Example 1 were determined by LC-MS/MS.

### Results

The brain and plasma concentrations of the compound of Example 1 in 4 rats given via an IV infusion are listed in Table 2. Plasma concentrations at 2 and 4 hours were within 2-fold, which suggests that steady state was achieved. The mean±SD total concentrations of the compound of Example 1 at 4 hours were 2.41±0.46 μM in plasma and 1.74±0.37 μM in brain. The mean brain versus plasma concentration ratio was 73%. The mean CSF concentration of the compound of Example 1 at 4 hours was 0.579±0.062 μM, which represents 43% of the estimated unbound plasma concentration at 4 hours. Unbound plasma concentrations of the compound of Example 1 were calculated using the in vitro rat plasma unbound fraction of 57%. These data suggest that the compound of Example 1 readily penetrates across the rat blood-brain barrier. This is consistent with in vitro permeability studies using Caco-2 cells, which showed that the compound of Example 1 is highly permeable (A-B Pₐₚₚ 16×10⁻⁶ cm/s) and is not a substrate of P-gp and BCRP efflux transporters.

TABLE 2
Brain, CSF, and Plasma Concentrations (μM) of the compound of Example
1 in Male Rats Administered Example 1 via an IV Infusion for 4 Hours

| | | | 1001 | 1002 | 1003 | 1004 | Mean |
|---|---|---|---|---|---|---|---|
| Plasma | Total | 2 h (μM) | 1.27 | 1.44 | 1.41 | 1.71 | 1.46 ± 0.18 |
| | | 4 h (μM) | 1.95 | 2.55 | 2.14 | 2.98 | 2.41 ± 0.46 |
| | Free^{a} | 4 h (μM) | 1.11 | 1.45 | 1.22 | 1.70 | 1.37 ± 0.26 |

| CSF | 4 h (μM) | 0.604 | 0.541 | 0.517 | 0.653 | 0.579 ± 0.062 |
|---|---|---|---|---|---|---|
| | CSF/unbound plasma | 54 | 37 | 42 | 38 | 43 |
| | concentration (%) | | | | | |
| Brain | 4 h (μM) | 1.36 | 1.52 | 1.91 | 2.17 | 1.74 ± 0.37 |
| | Brain/Plasma (%) | 70 | 60 | 89 | 73 | 73 |

^{a}Calculated using rat plasma in vitro unbound fraction of 72%

Various modifications of the invention, in addition to those described herein, will be apparent to those skilled in the art from the foregoing description. Ssuch modifications are also intended to fall within the scope of the appended claims. Each reference, including all patent, patent applications, and publications, cited in the present application is incorporated herein by reference in its entirety.

1. A compound of Formula (I): 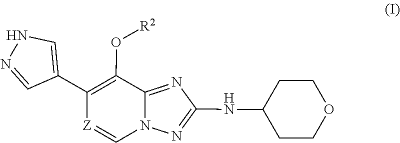
or pharmaceutically acceptable salts thereof, wherein:
Z is N or CH; and
R² is C₁₋₄ alkyl or C₁₋₄ haloalkyl.

2. The compound of claim 1, or a pharmaceutically acceptable salt thereof, wherein Z is N or CH; and R² is C₁₋₃ alkyl or C₁₋₃ haloalkyl.

3. The compound of claim 1, or a pharmaceutically acceptable salt thereof, wherein Z is N or CH; and R² is C₁₋₃ alkyl or C₁₋₃ fluoroalkyl.

4. The compound of claim 1, or a pharmaceutically acceptable salt thereof, wherein Z is N or CH; and R² is C₁₋₃ alkyl.

5. The compound of claim 1, or a pharmaceutically acceptable salt thereof, wherein Z is N or CH; and R² is C₁₋₃ fluoroalkyl.

6. The compound of claim 1, or a pharmaceutically acceptable salt thereof, wherein Z is N.

7. The compound of claim 1, or a pharmaceutically acceptable salt thereof, wherein Z is CH.

8. The compound of claim 1, wherein the compound is a compound of Formula (I): 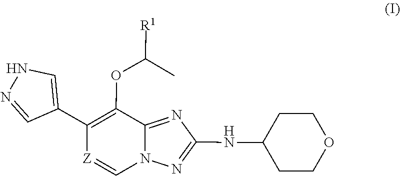
or a pharmaceutically acceptable salt thereof, wherein:
Z is CH or N; and
R¹ is selected from CH₃, CHF₂, and CF₃.

9. The compound of claim 8, or a pharmaceutically acceptable salt thereof, wherein R¹ is CH₃.

10. The compound of claim 8, or a pharmaceutically acceptable salt thereof, wherein R¹ is CHF₂.

11. The compound of claim 8, or a pharmaceutically acceptable salt thereof, wherein R¹ is CF₃.

12. The compound of claim 1, having Formula (II): 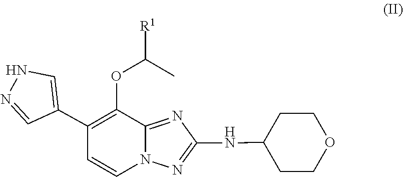
or a pharmaceutically acceptable salt thereof.

13. The compound of claim 1, having Formula (III): 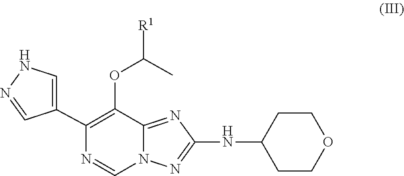
or a pharmaceutically acceptable salt thereof.

14. The compound of claim 8, or a pharmaceutically acceptable salt thereof, wherein Z is CH and R¹ is CH₃.

15. The compound of claim 8, or a pharmaceutically acceptable salt thereof, wherein Z is CH and R¹ is CHF₂.

16. The compound of claim 8, or a pharmaceutically acceptable salt thereof, wherein Z is CH and R¹ is CF₃.

17. The compound of claim 8, or a pharmaceutically acceptable salt thereof, wherein Z is N and R¹ is CH₃.

18. The compound of claim 8, or a pharmaceutically acceptable salt thereof, wherein Z is N and R¹ is CHF₂.

19. The compound of claim 8, or a pharmaceutically acceptable salt thereof, wherein Z is N and R¹ is CF₃.

20. The compound of claim 1, selected from:
8-isopropoxy-7-(1H-pyrazol-4-yl)-N-(tetrahydro-2H-pyran-4-yl)-[1,2,4]triazolo[1,5-c]pyrimidin-2-amine;
8-isopropoxy-7-(1H-pyrazol-4-yl)-N-(tetrahydro-2H-pyran-4-yl)-[1,2,4]triazolo[1,5-a]pyridin-2-amine;
(R)-7-(1H-pyrazol-4-yl)-N-(tetrahydro-2H-pyran-4-yl)-8-((1,1,1-trifluoropropan-2-yl)oxy)-[1,2,4]triazolo[1,5-a]pyridin-2-amine;
(S)-7-(1H-pyrazol-4-yl)-N-(tetrahydro-2H-pyran-4-yl)-8-((1,1,1-trifluoropropan-2-yl)oxy)-[1,2,4]triazolo[1,5-a]pyridin-2-amine;
(R)-8-((1,1-difluoropropan-2-yl)oxy)-7-(1H-pyrazol-4-yl)-N-(tetrahydro-2H-pyran-4-yl)-[1,2,4]triazolo[1,5-a]pyridin-2-amine;
(S)-8-((1,1-difluoropropan-2-yl)oxy)-7-(1H-pyrazol-4-yl)-N-(tetrahydro-2H-pyran-4-yl)-[1,2,4]triazolo[1,5-a]pyridin-2-amine;
(R)-7-(1H-pyrazol-4-yl)-N-(tetrahydro-2H-pyran-4-yl)-8-((1,1,1-trifluoropropan-2-yl)oxy)-[1,2,4]triazolo[1,5-c]pyrimidin-2-amine;
(S)-7-(1H-pyrazol-4-yl)-N-(tetrahydro-2H-pyran-4-yl)-8-((1,1,1-trifluoropropan-2-yl)oxy)-[1,2,4]triazolo[1,5-c]pyrimidin-2-amine;
(R)-8-((1,1-difluoropropan-2-yl)oxy)-7-(1H-pyrazol-4-yl)-N-(tetrahydro-2H-pyran-4-yl)-[1,2,4]triazolo[1,5-c]pyrimidin-2-amine; and
(S)-8-((1,1-difluoropropan-2-yl)oxy)-7-(1H-pyrazol-4-yl)-N-(tetrahydro-2H-pyran-4-yl)-[1,2,4]triazolo[1,5-c]pyrimidin-2-amine;
or a pharmaceutically acceptable salt thereof.

21. The compound of claim 1, which is 8-isopropoxy-7-(1H-pyrazol-4-yl)-N-(tetrahydro-2H-pyran-4-yl)-[1,2,4]triazolo[1,5-c]pyrimidin-2-amine, or a pharmaceutically acceptable salt thereof.

22. The compound of claim 1, or a pharmaceutically acceptable salt thereof, wherein the compound or salt is a blood brain barrier penetrant.

23. A pharmaceutical composition comprising the compound of claim 1, or a pharmaceutically acceptable salt thereof, and a pharmaceutically acceptable carrier.

24. A method of inhibiting CDK2, comprising contacting the CDK2 with the compound of claim 1, or a pharmaceutically acceptable salt thereof.

25. A method of inhibiting CDK2 in a patient, comprising administering to the patient the compound of claim 1, or a pharmaceutically acceptable salt thereof.

26. A method of treating a disease or disorder associated with CDK2 in a patient, comprising administering to the patient a therapeutically effective amount of the compound of claim 1, or pharmaceutically acceptable salt thereof.

27. The method of claim 26, wherein the disease or disorder is associated with an amplification of the cyclin E1 (CCNE1) gene and/or overexpression of CCNE1.

28. A method of treating a human subject having a disease or disorder associated with cyclin-dependent kinase 2 (CDK2), comprising administering to the human subject the compound of claim 1, or a pharmaceutically acceptable salt thereof, wherein the human subject has been previously determined to:
(i)
(a) have a nucleotide sequence encoding a p16 protein comprising the amino acid sequence of SEQ ID NO:1; and/or
(b) have a cyclin dependent kinase inhibitor 2A (CDKN2A) gene lacking one or more inactivating nucleic acid substitutions and/or deletions;
(ii)
(a) have an amplification of the cyclin E1 (CCNE1) gene; and/or
(b) have an expression level of CCNE1 in a biological sample obtained from the human subject that is higher than a control expression level of CCNE1.

29. A method of treating a human subject having a disease or disorder associated with cyclin-dependent kinase 2 (CDK2), comprising:
(i) identifying, in a biological sample obtained from the human subject:
(a) a nucleotide sequence encoding a p16 protein comprising the amino acid sequence of SEQ ID NO:1; and/or
(b) a cyclin dependent kinase inhibitor 2A (CDKN2A) gene lacking one or more inactivating nucleic acid substitutions;
(ii) identifying, in a biological sample obtained from the human subject:
(a) an amplification of the cyclin E1 (CCNE1) gene; and/or
(b) an expression level of CCNE1 that is higher than a control expression level of CCNE1; and
(iii) administering the compound of claim 1, or a pharmaceutically acceptable salt thereof, to the human subject.

30. The method of claim 29, comprising:
(i) identifying, in a biological sample obtained from the human subject:
(a) a nucleotide sequence encoding a p16 protein comprising the amino acid sequence of SEQ ID NO:1; and/or
(b) a CDKN2A gene lacking one or more inactivating nucleic acid substitutions and/or deletions;
(ii) identifying, in a biological sample obtained from the human subject:
(a) an amplification of the CCNE1 gene; and
(iii) administering the compound or the salt to the human subject.

31. A method of evaluating the response of a human subject having a disease or disorder associated with cyclin-dependent kinase 2 (CDK2) to the compound of claim 1, or a pharmaceutically acceptable salt thereof, comprising:
(a) administering the compound or the salt, to the human subject, wherein the human subject has been previously determined to have an amplification of the cyclin E1 (CCNE1) gene and/or an expression level of CCNE1 that is higher than a control expression level of CCNE1;
(b) measuring, in a biological sample of obtained from the subject subsequent to the administering of step (a), the level of retinoblastoma (Rb) protein phosphorylation at the serine corresponding to amino acid position 780 of SEQ ID NO:3,
wherein a reduced level of Rb phosphorylation at the serine corresponding to amino acid position 780 of SEQ ID NO:3, as compared to a control level of Rb phosphorylation at the serine corresponding to amino acid position 780 of SEQ ID NO:3, is indicative that the human subject responds to the compound or the salt.

32. The method of claim 26, wherein the disease or disorder is cancer.

33. The method of claim 32, wherein the cancer is a cancer that has metathesized to the brain.

34. The method of claim 33, wherein the cancer is breast cancer.

35. The method of claim 33, wherein the cancer is lung cancer.

36. The method of claim 32, wherein the cancer is metastatic breast cancer.

37. The method of claim 32, wherein the cancer is metastatic lung cancer.

38. The method of claim 37, wherein the metastatic lung cancer is metastatic non-small cell lung cancer.

39. The method of claim 37, wherein the metastatic lung cancer is metastatic small cell lung cancer.


SEQUENCE LISTING (3 sequences)

SEQ ID NO: 1 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 2 - protein, 410 aa, Homo sapiens
MPRERRERDAKERDTMKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 3 - protein, 928 aa, Homo sapiens
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK